(19) United States
(12) Patent Application Publication
(10) Pub. No.: US 2016/0202247 A1
(43) Pub. Date: Jul. 14, 2016

(54) PHOTO-PATTERNABLE OPTICAL LUMINESCENCE DUAL SENSORS AND METHODS OF PREPARING AND USING THEM

(71) Applicants: Yanqing Tian, Tempe, AZ (US); Hong Wang, Tempe, AZ (US); Ganquan Song, Mesa, AZ (US); Fengyu Su, Tempe, AZ (US); Liqiang Zhang, Chandler, AZ (US); Deirdre Meldrum, Phoenix, AZ (US); Laimonas Kelbauskas, Gilbert, AZ (US); Benjamin Ueberroth, Tempe, AZ (US)

(72) Inventors: Yanqing Tian, Tempe, AZ (US); Hong Wang, Tempe, AZ (US); Ganquan Song, Mesa, AZ (US); Fengyu Su, Tempe, AZ (US); Liqiang Zhang, Chandler, AZ (US); Deirdre Meldrum, Phoenix, AZ (US); Laimonas Kelbauskas, Gilbert, AZ (US); Benjamin Ueberroth, Tempe, AZ (US)

(21) Appl. No.: 14/996,170

(22) Filed: Jan. 14, 2016

Related U.S. Application Data
(60) Provisional application No. 62/103,527, filed on Jan. 14, 2015.

(51) Int. Cl. G01N 33/52; G01N 21/64

(52) U.S. Cl. CPC G01N 33/523; G01N 21/6428; G01N 2021/6439

(57) ABSTRACT

The present disclosure relates to an optical luminescence dual sensor comprising a polymerized form of a probe for sensing pH; a polymerized form of a probe for sensing oxygen; a polymerized form of an internal reference probe; and a matrix. The present disclosure also relates to methods of preparing an optical luminescence dual sensor and methods of using them.

### FEDERALLY SPONSORED RESEARCH OR DEVELOPMENT

This invention was made with government support from grant U01 CA164250 and P50 HG002360 awarded by the National Institutes of Health. The government has certain rights in the invention.

### FIELD OF THE INVENTION

The present disclosure relates to an optical luminescence dual sensor comprising a polymerized form of a probe for sensing pH; a polymerized form of a probe for sensing oxygen; a polymerized form of an internal reference probe; and a matrix. The present disclosure also relates to methods of preparing an optical luminescence dual sensor and methods of using them.

### SUMMARY OF THE INVENTION

The present disclosure provides an optical luminescence dual sensor having three emission colors. In particular, the optical luminescence dual sensors comprise a polymerized form of a probe for sensing pH; a polymerized form of a probe for sensing oxygen; a polymerized fonn of an internal reference probe; and a matrix.

The probe for sensing pH has formula 1:

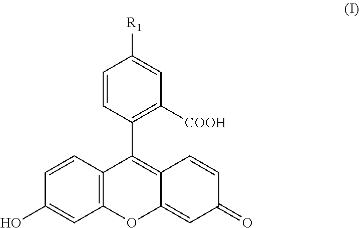

wherein
R₁ is CₘH₂ₘX or NHCOCₘH₂ₘY, where m is an integer selected from the group consisting of 0, 1, 2, 3, 4, 5, 6, 8 and 11;
X is selected from the group consisting of:

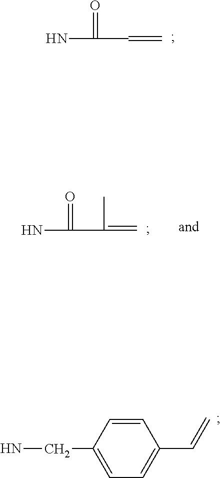

and
Y is selected from the group consisting of:

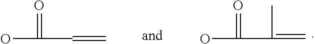

The probe for sensing oxygei has formula II:

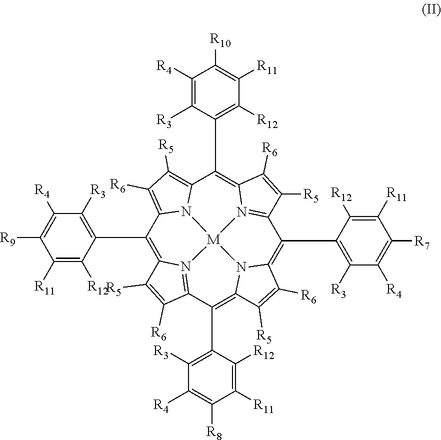

wherein

M is selected from platinum (Pt) or palladium (Pd);

R₁₁ and R₁₂ can be the same or different and are independently selected from the group consisting of H, halo, CH₃, OCH₃ and OC₂H₅;

R₃ and R₄ can be the same or different and are independently selected from the group consisting of H, halo, CH₃, OCH₃ and OC₂H₅;

R₅ and R₆ can be the same or different and are independently selected from the group consisting of H, halo, CH₃, OCH₃ and OC₂H₅;

R₇, R₈, R₉ and R₁₀ can be the same or different and are independently selected from the group consisting of (CH₂)ₚOH, O(CH₂)ₚOH, NH(CH₂)ₚOH, (CH₂)ₚOM′A, O(CH₂)ₚOM′A, NH(CH₂)ₚOM′A, (CH₂)ₚOA, O(CH₂)ₚOA, NH(CH₂)ₚOA, (CH₂)ₚOVA, O(CH₂)ₚOVA, NH(CH₂)ₚOVA, (OCH₂CH₂)_{q}OH, NH(CH₂CH₂O)_{q}H, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA, NH(CH₂CH₂O)_{q}VA, where

M′A is

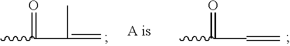

### VA is

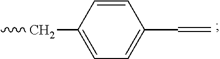

p is an integer selected from the group of consisting of 0, 1, 2, 3, 4, 5, 6, 7, 8, 9, 10, 11 and 12; and q is an integer selected from the group of consisting of 1, 2, 3, 4, 5, 6, 7, 8, 9, 10, 11, 12, 13, 14, 15, 16, 17, 18, 19, 20, 21, 22, 23, 38, 39, 40, 41, 42, 43, 44, 140, 141, 142, 143, 144, 145, 146, 147, 148, 149 and 150.

The internal reference probe has formula

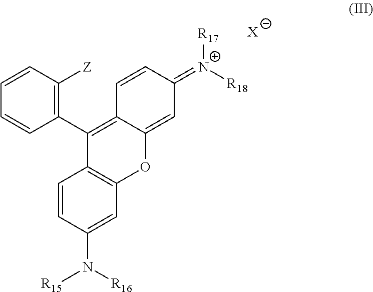

wherein R₁₅, R₁₆, R₁₇, and R₁₈ can be the same or different and are independently CₙH₂ₙ₊₁, where n is an integer selected from the group consisting of 1, 2, 3, 4, 5, 6, 7 and 8;

X is an anion;

Z is selected from the group consisting of: (CH₂)ₚOH, O(CH₂)ₚOH, NH(CH₂)ₚOH, (CH₂)ₚOM′A, O(CH₂)ₚOM′A, NH(CH₂)ₚOM′A, (CH₂)ₚOA, O(CH₂)ₚOA, NH(CH₂)ₚOA, (CH₂)ₚOVA, O(CH₂)ₚOVA, NH(CH₂)ₚOVA, (OCH₂CH₂)_{q}OH, NH(CH₂CH₂O)_{q}H, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA, NH(CH₂CH₂O)_{q}VA, CH₂(OCH₂CH₂)ᵣOA, CH₂(OCH₂CH₂)ᵣOM′A, CH₂(OCH₂CH₂)ᵣOVA; and

r is an integer selected from the group of consisting of 1, 2, 3, 4, 5, 6, 7, 8, 9, 10, 11, 12, 13, 14, 15, 16, 17, 18, 19, 20, 21, 22, 23, 38, 39, 40, 41, 42, 43, 44, 140, 141, 142, 143, 144, 145, 146, 147, 148, 149 and 150.

The present disclosure also provides a method of preparing an optical luminescence dual sensor. In the first step of the method, a probe for sensing pH, a probe for sensing oxygen, and an internal reference probe are copolymerized with polyacrylamide, and poly(2-hydroxyethyl methacrylate -o-polyacryiamide (PHEMA-co-PAK) in the presence of a crossiinker and an initiator.

The present disclosure also provides a dual pH and oxygen sensor pattern produced by the method of preparing an array of dual pH and oxygen sensors on a substrate.

The present disclosure also provides a method of determining the pH of a sample. The method comprises (a) exposing the sample to an optical luminescence dual sensor as defined above; (b) irradiating the sensor at a first wavelength to produce a pH indicator emission signal at a second wavelength and an internal reference emission signal at a third wavelength; (c) measuring the pH indicator emission signal at the second wavelength; (d) measuring the internal reference emission signal at the third emission wavelength; and (e) ratiometrically detemiining the pH of the sample.

The present disclosure also provides a method of determining oxygen concentration in a sample. The method comprises (a) exposing the sample to an optical luminescence dual sensor as defined above; (b) irradiating the sensor at a first wavelength to produce an oxygen indicator emission signal at a second wavelength and an internal reference emission signal at a third wavelength; (c) measuring the oxygen indicator emission signal at the second wavelength; (d) measuring the internal reference emission signal at the third wavelength; and (e) ratiometrically determining the oxygen concentration in the sample.

The present disclosure also provides a method of simultaneously determining the pH and oxygen concentration in a sample. The method comprises (a) exposing the sample to an optical luminescence dual sensor as defined above; (b) irradiating the sensor (i) at a first wavelength to produce a pH indicator emission signal at a second wavelength, (ii) at a third wavelength to produce an oxygen indicator emission signal at a fourth wavelength and (iii) at a fifth wavelength to produce an internal reference emission signal at a sixth wavelength; (c) measuring the pH indicator emission signal at the second wavelength; (d) measuring the oxygen indicator emission signal at the fourth wavelength; (e) measuring the internal reference emission signal at the sixth wavelength; (f) ratiometrically determining the pH of the sample using the measurements obtained in steps (c) and (e); and (g) ratiometrically determining the oxygen concentration of the sample using the measurements obtained in steps (d) and (e).

The present disclosure also provides a method of detecting cellular respiration in a sample. The method comprises: (a) exposing the cell to an optical luminescence dual sensor as defined above; (b) irradiating the sensor at a first wavelength to produce an oxygen indicator emission signal at a second wavelength and an internal reference emission signal at a third wavelength at a first time point; (c) measuring the oxygen indicator emission signal at the second wavelength; (d) measuring the internal reference emission signal at the third wavelength; (e) ratiometrically determining the oxygen concentration in the sample; and (f) repeating steps (b)-(e) at least at a second time point. A decrease in the oxygen concentration at the at least second time point indicates cell respiration in the sample.

The present disclosure also provides a method of detecting single cell respiration. The method comprises: (a) exposing the cell to an optical luminescence dual sensor as defined above; (b) irradiating the sensor at a first wavelength to produce an oxygen indicator emission signal at a second wavelength and an internal reference emission signal at a third wavelength at a first time point; (c) measuring the oxygen indicator emission signal at the second wavelength; (d) measuring the internal reference emission signal at the third wavelength; (e) ratiometrically determining the oxygen concentration in the sample; and (f) repeating steps (b)-(e) at least at a second time point. A decrease in the oxygen concentration at the at least second time point indicates cell respiration.

The present disclosure also provides a method of determining a cellular respiration rate in a sample. The method comprises: (a) exposing the sample to an optical luminescence dual sensor as defined above; (b) irradiating the sensor at a first wavelength to produce an oxygen indicator emission signal at a second wavelength and an internal reference emission signal at a third wavelength at a first time point; (c) measuring the oxygen indicator emission signal at the second wavelength; (d) measuring the internal reference emission signal at the third wavelength; (e) ratiometricaily determining the oxygen concentration in the sample; and (f) repeating steps (b)-(e) at least at a second time point, (g) determining the respiration rate from the difference in oxygen concentration in the sample at the first time point and the at least second time point as a function of time.

The present disci. sure also provides a method of detecting extracellular acidification in a sample. The method comprises: (a) exposing the sample to an optical luminescence dual sensor as defined above; (b) irradiating the sensor at a first wavelength to produce a pH indicator emission signal at a second wavelength and an internal reference emission signal at a third wavelength at a first time point; (c) measuring the indicator emission signal at the second wavelength; (d) measuring the internal reference emission signal at the third wavelength; (e) ratiometrically determining the pH in the sample; and (f) repeating steps (b)-(e) at least at a second time point. A decrease in the pH at the at least second time point indicates extracellular acidification.

The present disclosure also provides a method of detecting extracellular acidification of a single cell. The method comprises: (a) exposing the cell to an optical luminescence dual sensor as defined above; (b) irradiating the sensor at a first wavelength to produce a pH indicator emission signal at a second wavelength and an internal reference emission signal at a third wavelength at a first time point; (c) measuring the indicator emission signal at the second wavelength; (d) measuring the internal reference emission signal at the third wavelength; (e) ratiometrically determining the pH in the sample; and (f) repeating steps (b)-(e) at least at a second time point. A decrease in the pH at the at least second time point indicates extracellular acidification.

The present disclosure also provides a method of determining extracellular acidification rate in a sample. The method comprises: (a) exposing the sample to an optical luminescence dual sensor as defined above; (b) irradiating the sensor at a first wavelength to produce a pH indicator emission signal at a second wavelength and an internal reference emission signal at a third wavelength at a first time point; (c) measuring the pH indicator emission signal at the second wavelength; (d) measuring the internal reference emission signal at the third wavelength; (e) ratiometrically determining the pH in the sample; (f) repeating steps (b)-(e) at least at a second time point; and (g) determining the extracellular acidification rate from the difference in pH in the sample at the first time point and the at least second time point as a function of time.

The present disclosure also provides a method of simultaneously determining the pH and oxygen concentration in a single cell. The method comprises (a) exposing the cell to an optical luminescence dual sensor as defined above; (b) irradiating the sensor (i) at a first wavelength to produce a pH indicator emission signal at a second wavelength, (ii) at a third wavelength to produce an oxygen indicator emission signal at a fourth wavelength and (iii) at a fifth wavelength to produce an internal reference emission signal at a sixth wavelength; (c) measuring the pH indicator emission signal at the second wavelength; (d) measuring the oxygen indicator emission signal at the fourth wavelength; (e) measuring the internal reference emission signal at the sixth wavelength; (f) ratiometrically determining the pH of the sample using the measurements obtained in steps (c) and (e); (g) ratiometricaily determining the oxygen concentration of the sample using the measurements obtained in steps (d) and (e); and (h) repeating steps (b)-(g) at least at a second time point. A decrease in the oxygen concentration at the at least second time point indicates cell respiration and a decrease in the pH at the at least second time point indicates extracellular acidification.

### BRIEF DESCRIPTION OF THE FIGURES

FIG. 1 shows a schematic diagram for preparing an optical luminescence dual sensor according to an embodiment of the invention.

FIG. 2A shows pH responses of a pH sensor film excited at 488 ram FIG. 2B shows pH responses as measured using emission intensity 15 mm. I is the intensity at 515 nm. I₀ is the intensity at 515 nm at pH 3.

FIG. 3A shows the responses of an oxygen sensor film to changes in dissolved oxygen concentration. FIG. 3B shows the Stern-Volmer plot of an oxygen sensor at different dissolved oxygen concentration.

FIG. 4A shows the response of a dual sensor according to an embodiment of the invention (excitation at 488 nm). FIG. 4B shows responses of the reference probe (left peak) and oxygen probe (right peak). FIG. 4C shows pH responses as measured using emission intensity at 515 nm normalized against the intensity value at pH 3 (I₀), and the ratio between the emission intensities at 515 nm and 580 nm. FIG. 4D shows oxygen responses excited at 405 nm. FIG. 4E shows the response of the dual sensor to changes in oxygen concentration (excitation at 540 nm). FIG. 4F shows Stern-Volmer plots of the oxygen responses using different methods. Note dissolved oxygen in air saturated water at 23° C. is 8.6 mg/L or 8.6 ppm.

FIG. 5 shows micro-wells with loaded single cells (scale bar: 100 μm).

FIG. 6 shows a schematic of a device for single cell analysis—: “Draw-down” configuration.

FIG. 7A-FIG. 7D show pH and oxygen responses of the tri-color dual pH and oxygen sensor in 3×3 micropatterned arrays. FIG. 7A shows Stern-Volmer graphs of the oxygen sensors response to changes in dissolved oxygen concentration, FIG. 7B shows: pH responses. FIG. 7C shows the response of the internal reference—rhodamine—to changes in dissolved oxygen concentration. FIG. 7D shows the the response of the internal reference—rhodamine—to changes in pH. Oxygen sensor was excited at 386 nm and its emission signal was collected at 647 nm. pH sensor was excited at 470 nm and its emission signal was collected at 528 nm. Rhodamine fluorescence (internal reference was excited at 525 nm and its emission was collected at 575 nm.

FIG. 8A-FIG. 8B show heterogeneity in the oxygen consumption (FIG. 8A) and extracellular acidification rates (FIG. 8B) among individual mammalian cells.

### DETAILED DESCRIPTION OF THE INVENTION

In order that the invention herein described may be fully understood, the following detailed description is set forth.

The invention includes the following:
(1) An optical luminescence dual sensor comprising a copolymer, wherein the copolymer comprises:

(a) a polymerized form of a probe for sensing pH;

(b) a polymerized form of a probe for sensing oxygen;

(c) a polymerized form of an internal reference probe; and

(d) a matrix comprising a polymer selected from the group consisting of poly(2-hydroxyethyl methacrylate) (PHEMA), polyacryiamide (PAM), poly(poly(2-(2-(2-methoxyethoxy)ethoxy)ethyl methacrylate)) (POEGMA), poly(N-isopropyl acrylamide) (PNIPAAm); and copolymers thereof;

wherein:

the probe for sensing pH has formula (I):

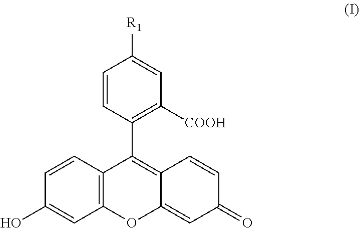

wherein
R₁ is CₘH₂ₘX or NHCOCₘH₂ₘY, where m is an integer selected from the group consisting of 0, 1, 2, 3, 4, 5, 6, 8 and 11;
X is selected from the group consisting of:

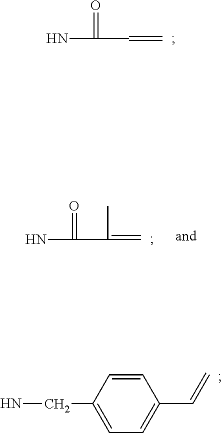

and
Y is selected from the group consisting of:

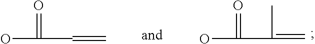

the probe for sensing oxygen has formula

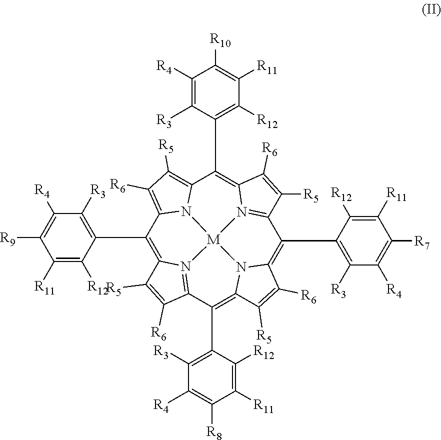

wherein
M is selected from Pt or Pd;
R₁₁ and R₁₂ can be the same or different and are independently selected from the group consisting of H, halo, CH₃, OCH₃ and OC₂H₅;
R₃ and R₄ can be the same or different and are independently selected from the group consisting of H, halo, CH₃, OCH₃ and OC₂H₅;
R₅ and R₆ can be the same or different and are independently selected from the group consisting of H, halo, CH₃, OCH₃ and OC₂H₅;
R₇, R₈, R₉ and R₁₀ can be the same or different and are independently selected from the group consisting of (CH₂)ₚOH, O(CH₂)ₚOH, NH(CH₂)ₚOH, (CH₂)ₚOM′A, O(CH₂)ₚOM′A, NH(CH₂)ₚOM′A, (CH₂)ₚOA, O(CH₂)ₚOA, NH(CH₂)ₚOA, (CH₂)ₚOVA, O(CH₂)ₚOVA, NH(CH₂)ₚOVA, (OCH₂CH₂)_{q}OH, NH(CH₂CH₂O)_{q}H, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA, NE(CH₂CH₂O)_{q}VA,
where
   M′A is

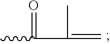

A is

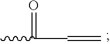

VA is

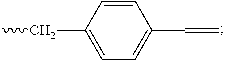

p is an integer selected from the group of consisting of 0, 1, 2, 3, 4, 5, 6, , 9, 10, 11 and 12; and
q is an integer selected from the group of consisting of 1, 2, 3, 4, 5, 6, 7, 8, 9, 10, 11, 12, 13, 14, 15, 16, 17, 18, 19, 20, 21, 22, 23, 38, 39, 40, 41, 42, 43, 44, 140, 141, 142, 143, 144, 145, 146, 147, 148, 149 and 150; and

the internal reference probe has formula (III):

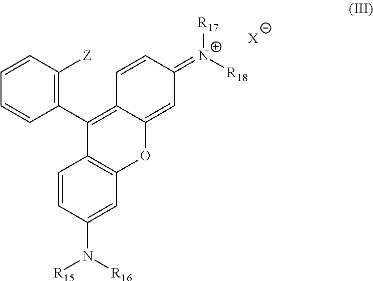

wherein
R₁₅, R₁₆, R₁₇, and R₁₈ can he the same or different and are independently CₙH₂ₙ₊₁, where n is an integer selected from the group consisting of 1, 2, 3, 4, 5, 6, 7 and 8;
X is an anion;
Z is selected from the group consisting of: (CH₂)ₚOH, O(CH₂)ₚOH, NH(CH₂)ₚOH, (CH₂)ₚOM′A, O(CH₂)ₚOM′A, NH(CH₂)ₚOM′A, (CH₂)ₚOA, O(CH₂)ₚOA, NH(CH₂)ₚOA, (CH₂)ₚOVA, O(CH₂)ₚOVA, NH(CH₂)ₚOVA, (OCH₂CH₂)_{q}OH, NH(CH₂CH₂O)_{q}H, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA, NH(CH₂CH₂O)_{q}VA, CH₂(OCH₂CH₂)ᵣOA, CH₂(OCH₂CH₂)ᵣOM′A, CH₂(OCH₂CH₂)ᵣOVA; and
r is an integer selected from the group of consisting of 1, 2, 3, 4, 5, 6, 7, 8, 9, 10, 11, 12, 13, 14, 15, 16, 17, 18, 19, 20, 21, 22, 23, 38, 39, 40, 41, 42, 43, 44, 140, 141, 142, 143, 144, 145, 146, 147, 148, 149 and 150.
(2) The optical luminescence dual sensor of the above (1), wherein, in the probe for sensing pH, R₁ is CₘH₂ₘX, where m is 0, 1, 2 or 3.
(3) The optical luminescence dual sensor of the above (1) or (2), wherein, in the probe for sensing pH, R₁ is CₘH₂ₘX, where m is 0.
(4) The optical luminescence dual sensor of any of the above (1)-(3), wherein, in the probe for sensing pH, X is

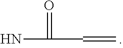

(5) The optical luminescence dual sensor of any of the above (1)-(4), wherein the probe for sensing pH is:

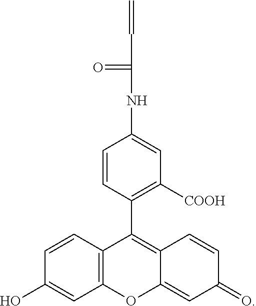 [SMILES: C=CC(=O)Nc1ccc(-c2c3ccc(=O)cc-3oc3cc(O)ccc23)c(C(=O)O)c1]

(6) The optical luminescence dual sensor according to any of the above (1)-(5), wherein, in the probe for sensing oxygen, R₃, R₄, R₅, R₆, R₁₁ and R₁₂ are independently halo or H.
(7) The optical luminescence dual sensor according to any of the above (1)-(6), wherein, in the probe for sensing oxygen, R₃, R₄, R₅, R₆, R₁₁ and R₁₂ are independently F or H.
(8) The optical luminescence dual sensor according to any of the above (1)-(7), wherein, in the probe for sensing oxygen, R₃, R₄, R₁₁ and R₁₂ are F; and R₅ and R₆ are H.
(9) The optical luminescence dual sensor according to any of the above (1)-(8), wherein, in the probe for sensing oxygen, M is Pt.
(10) The optical luminescence dual sensor according to any of the above (1)-(9), wherein, in the probe for sensing oxygen, R₇, R₈, R₉ and R₁₀ are O(CH₂)ₚOM′A.
(11) The optical luminescence dual sensor according to any of the above (1)-(10), wherein, in the probe for sensing oxygen, p is 2.
(12) The optical luminescence dual sensor according to any one of the above (1)-(11), wherein the probe for sensing oxygen is:

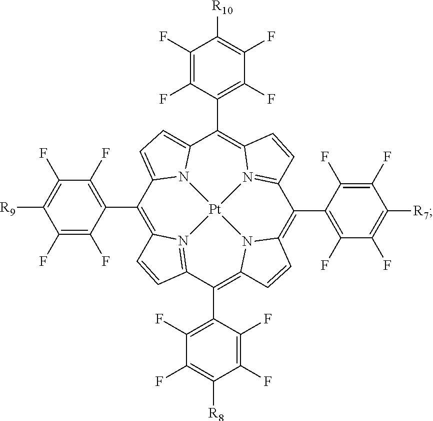

and

R₇, R₈, R₉ and R₁₀ are

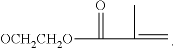

(13) The optical luminescence dual sensor according to any one of the above (1)-(12), wherein, in the internal reference probe, R₁₅, R₁₆, R₁₇, R₁₈ are CₙH₂ₙ₊₁, where n is an integer selected from the group consisting of 1, 2, 3, 4, 5 and 6.
(14) The optical luminescence dual sensor according to any one of the above (1)-(13), wherein, in the internal reference probe, R₁₅, R₁₆, R₁₇, R₁₈ are CₙH₂ₙ₊₁, where n is an integer selected from the group consisting of 1, 2 and 3.
(15) The optical luminescence dual sensor according to any one of the above (1)-(14), wherein, in the internal reference probe, R₁₅, R₁₆, R₁₇, R₁₈ are CₙH₂₊₁, where n is 2.
(16) The optical luminescence dual sensor according to any one of the above (1)-(15), wherein, in the internal reference probe, X is halo.
(17) The optical luminescence dual sensor according to any one of the above (1)-(16), wherein, in the internal reference probe, X is Cl.
(18) The optical luminescence dual sensor according to any one of the above (1)-(17), wherein, in the internal reference probe, Z is (CH₁)ₚOM′A.
(19) The optical luminescence dual sensor according to any one of the above (1)-(18), wherein, in the internal reference probe, p is 1.
(20) The optical luminescence dual sensor according to any one of the above (1)-(19), wherein the internal reference probe is:

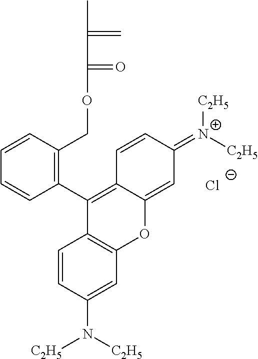

(21) The optical luminescence dual sensor according to any one of the above (1)-(20), further comprising a substrate.
(22) The optical luminescence dual sensor of the bove (21), wherein the substrate is selected from the group consisting of quartz glass, fused silica, silica particles, silica gels, and poly(ethylene terephthalate).
(23) The optical luminescence dual sensor of the above (22), wherein the copolymer is immobilized or attached to the substrate.
(24) The optical luminescence dual sensor of the above (23), wherein the copolymer is attached to the substrate using a conjugating layer.
(25) The optical luminescence dual sensor of the above (24), wherein the conjugating layer comprises a silane.
(26) The optical luminescence dual sensor of the above (25), wherein the silane is a trimethoxysilane.
(27) The optical luminescence dual sensor of the above em n the silane is 3-acryloxypropyl trimethoxysilane.
(28) The optical luminescence dual sensor of any of the above (23)-(27), wherein the copolymer is photopatterned on the substrate.
(29) A method of preparing an luminescence dual sensor, wherein the method comprises the steps of:

(a) copolymerizing a probe for sensing pH, a probe for sensing oxygen, and an internal reference probe, with polyacrylamide and poly(2-hydroxyethyl methacrylate)-co-polyacrylamide in the presence of a crosslinker and an initiator;

wherein the probe for sensing pH has formula (1):

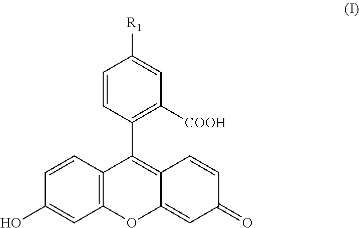

wherein
   RI is CₘH₂ₘX or NHCOCₘH₂ₘY, where m is an integer selected from the group consisting of 0, 1, 2, 3, 4, 5, 6, 8 and 11;
   X is selected from the group consisting of:

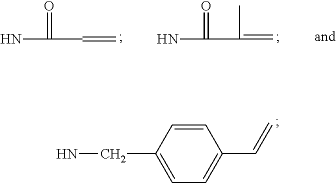

and
Y is selected from the group consisting of:

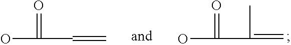

the probe for sensing oxygen has formula OD:

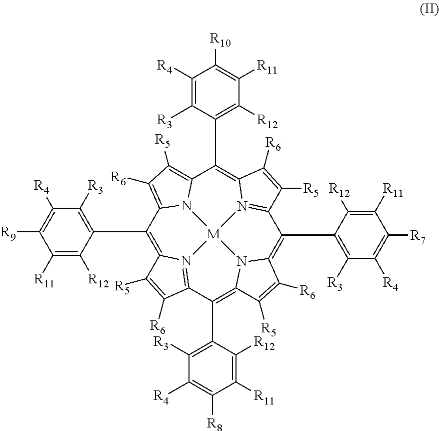

where M is selected from Pt or Pd;
   R₁₁ and R₁₂ can be the same or different and are independently selected from e group consisting of H, halo, CH₃, OCH₃ and OC₂H₅;
   R₃ and R₄ can be the same or different and are independently selected from the group consisting of H, halo, CH₃, OCH₃ and OC₂H₅;
   R₅ and R₆ can be the same or different and arc independently selected from the group consisting of H, halo, CH₃, OCH₃ and OC₂H₅;
   R₇, R₈, R₉ and R₁₀ can be the same or different and are independently selected from the group consisting of (CH₂)ₚOH, O(CH₂)ₚOH, NH(CH₂)ₚOH, (CH₂)ₚOM′A, O(CH₂)ₚOM′A, NH(CH₂)ₚOM′A, (CH₂)ₚOA, O(CH₂)ₚOA, NH(CH₂)ₚOA, (CH₂)ₚOVA, O(CH₂)ₚOVA, NH(CH₂)ₚOVA, (OCH₂CH₂)_{q}OH, NH(CH₂CH₂O)_{q}H, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA, NH(CH₂CH₂O)_{q}VA,
   where
      M′A is

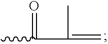

A is

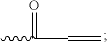

VA is

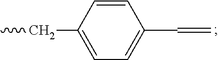

and
p is an integer selected from the group of consisting of 0, 1, 2, 3, 4, 5, 6, 7, 8, 9, 10, 11 and 12.
q is an integer selected from the group of consisting of 1, 2, 3, 4, 5, 6, 7, 8, 9, 10, 11, 12, 13, 14, 15, 16, 17, 18, 19, 20, 21, 22, 23, 38, 39, 40, 41, 42, 43, 44, 140, 141, 142, 143, 144, 145, 146, 147, 148, 149 and 150; and

the internal reference probe has formula MD:

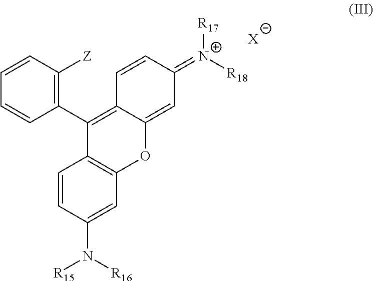

wherein R₁₅, R₁₆, R₁₇, and R₁₈ can be the same or different and are independently CₙH₂ₙ₊₁, where n is an integer selected from the group consisting of 1, 2, 3, 4, 5, 6, 7 and 8;
X is an anion; and
Z is selected from the group consisting of: (CH₂)ₚOH, O(CH₂)ₚOH, NH(CH₂)ₚOH, (CH₂)ₚOM′A, O(CH₂)ₚOM′A, NH(CH₂)ₚOM′A, (CH₂)ₚOA, O(CH₂)ₚOA, NH(CH₂)ₚOA, (CH₂)ₚOVA, O(CH₂)ₚOVA, NH(CH₂)ₚOVA, (OCH₂CH₂)_{q}OH, NH(CH₂CH₂O)_{q}H, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA, NH(CH₂CH₂O)_{q}VA, CH₂(OCH₂CH₂)ᵣOA, CH₂(OCH₂CH₂)ᵣOM′A, CH₂(OCH₂CH₂)ᵣOVA; and
r is an integer selected from the group of consisting of 1, 2, 3, 4, 5, 6, 7, 8, 9, 10, 11, 12, 13, 14, 15, 16, 17, 18, 19, 20, 21, 22, 23, 38, 39, 40, 41, 42, 43, 44, 140, 141, 142, 143, 144, 145, 146, 147, 148, 149 and 150
and

(b) immobilizing or attaching the copolymer of step (a) onto a substrate.
(30) The method of the above (29), wherein, in the probe for sensing pH, is R₁ is CₘH₂ₘX, where m is 0, 1, 2 or 3.
(31) The method of the above (29) or (30), wherein, in the probe for sensing pH, is R₁ is CₘH₂ₘX, where m is 0.
(32) The method of any of the above (29)-(31), erein, in the probe for sensing pH, X is

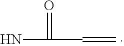

(33) The method of any of the above (29)-(32), wherein the probe for sensing pH is:

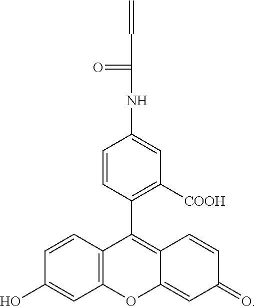 [SMILES: C=CC(=O)Nc1ccc(-c2c3ccc(=O)cc-3oc3cc(O)ccc23)c(C(=O)O)c1]

(34) The method of any of the above (29)-(33), wherein, in the probe for sensing oxygen, R₃, R₄, R₅, R₆, R₁₁ and R₁₂ are independently halo or H.
(35) The method of any of the above (29)-(34), wherein, in the probe for sensing oxygen, R₃, R₄, R₅, R₆, R₁₁ and R₁₂ are independently F or H.
(36) The method of any of the above (29)-(35), wherein, in the probe for sensing oxygen, R₃, R₄, R₁₁ and R₁₂ are F; and R₅ and R₆ are H.
(37) The method of any of the above (29)-(36), wherein, in the probe for sensing oxygen, M is Pt.
(38) The method of any of the above (29)-(37), wherein, in the probe for sensing oxygen, R₇, R₈, R₉ and R₁₀ are O(CH₂)ₚOM′A.
(39) The method of any of the above (29)-(38), wherein, in the probe for sensing oxygen, p is 2.
(40) The method of any of the above (29)-(39), wherein the probe for sensing oxygen is:

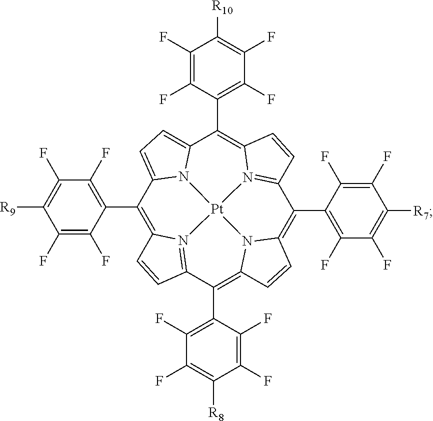

wherein R₇, R₈, R₉ and R₁₀ are

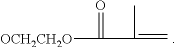

(41) The method of any of the above (29)-(40), wherein, in the internal reference probe, R₁₅, R₁₆, R₁₇, R₁₈ are CₙH₂ₙ₊₁, where n is an integer selected from the group consisting of 1, 2, 3, 4, 5 and 6.
(42) The method of any of the above (29)-(41), wherein, in the internal reference probe, R₁₅, R₁₆, R₁₇, R₁₈ are CₙH₂ₙ₊₁, where n is an integer selected from the group consisting of 1, 2 and 3.
(43) The method of any of the above (29)-(42), wherein, in the internal reference probe, R₁₅, R₁₆, R₁₇, R₁₈ are CₙH₂ₙ₊₁, where n is 2.
(44) The method of any of the above (29)-(43), wherein, in the internal reference probe, X is halo.
(45) The method of any of the above (29)-(44), wherein, in the internal reference probe, X is Cl.
(46) The method of any of the above (29)-(45), wherein, in the internal reference probe, Z is (CH₂)ₚOM′A.
(47) The method of any of the above (29)-(46), wherein, in the internal reference probe, p is 1.
(48) The method of any of the above (29)-(47), wherein the internal reference probe is:

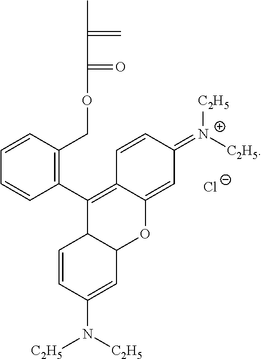 [SMILES: C=C(C)C(=O)OCc1ccccc1C1=C2C=CC(=[N+](CC)CC)C=C2OC2C=C(N(CC)CC)C=CC12 ; [Cl-]]

(49) The method of any of the above (29)-(48), wherein the crosslinker is selected from the group consisting of SR454, poly(ethylene glycol) diacrylate and poly(ethylene glycol) dimethacrylate.
(50) The method of any of the above (29)-(49), wherein the initiator is a photo-initiator.
(51) The method of the above (50), wherein the photo-initiator is selected from the group consisting of IRACURE® 819, 4-phenyl benzophenone, methyl o-benzoyl benzoate and benzyl dimethyl ketal.
(52) The method of any of the above (29)-(49), wherein the initiator is a thermal initiator.
(53) The method of the above (52), wherein the thermal initiator is AIBN or BPO.
(54) The method of any of the above (29)-(53), wherein the copolymer of step a attached to the substrate using a conjugating layer.
(55) The method of the above (54), wherein the conjugating layer comprises a silane.
(56) The method of the above (55), wherein the silane is a trimethoxysilane.
(57) The method of the above (56), wherein the silane is 3-acryloxypropyl trimethoxysilane.
(58) The method of any of the above (29)-(57), wherein the substrate is selected from the group consisting of quartz glass, fused silica, silica particles, silica gels, and poly(ethylene terephthalate)
(59) A method of preparing a dual pH and oxygen array on a substrate, wherein the method comprises:

(a) masking the substrate to define boundaries on the substrate;

(b) contacting the unmasked substrate with a conjugate compound to form a conjugated layer-substrate;

(c) contacting the conjugated layer-substrate with the sensor of any of the above (1)-(9).
(60) The method of the above (59), the conjugating layer comprises a silane.
(61) The method of the above (60), wherein the silane is a trimethoxysilane.
(62) The method of the above (61), wherein the silane is 3-acryloxypropyl trimethoxysilane.
(63) The method of any of the above (59)-(62), wherein the substrate is a fused silica surface.
(64) The method of any of the above (59)-(63), wherein step (c) comprises polymerizing:

(i) a probe for sensing pH;

(ii) a probe for sensing oxygen;

(iii) an internal reference probe; and

(iv) a matrix.
(65) The method of the above (64), wherein the polymerization step is in the presence of an initiator.
(66) The method of the above (65), wherein the initiator is a photo-initiator.
(67) The method of the above (65), wherein the initiator is a thermal initiator.
(68) The dual pH and oxygen sensor pattern roduced by the method of any of the above (59)-(67).
(69) A method of determining pH of a sample, wherein the method comprises:

(a) exposing the sample to an optical luminescence dual sensor according to any of the above (1)-(19);

(b) irradiating the sensor at a first wavelength to produce a pH indicator emission signal at a second wavelength and an internal reference emission signal at a third wavelength;

(c) measuring the pH indicator emission signal at the second wavelength;

(d) measuring the internal reference emission signal at the third emission wavelength; and

(e) ratiometrically determining the of the sample.
(70) A method of determining oxygen concentration in a sample, wherein the method comprises:

(a) exposing the sample to an optical luminescence dual sensor according to any of the above (1)-(19);

(b) irradiating the sensor at a first wavelength to produce an oxygen indicator emission signal at a second wavelength and an internal reference emission signal at a third wavelength;

(c) measuring the oxygen indicator emission signal at the second wavelength;

(d) measuring the internal reference emission signal at the third wavelength; and

(e) ratiometrically determining the oxygen concentration in the sample.
(71) A method of simultaneously determining pH and oxygen concentration in a sample, wherein the method comprises:

(a) exposing the sample to an optical luminescence dual sensor according to any of the above (1)-(19);

(b) irradiating the sensor (i) at a first wavelength to produce a pH indicator emission signal at a second wavelength, (ii) at a third wavelength to produce an oxygen indicator emission signal at a fourth wavelength and (iii) at a fifth wavelength to produce an internal reference emission signal at a sixth wavelength;

(c) measuring the pH indicator emission signal at the second wavelength;

(d) measuring the oxygen indicator emission signal at the third wavelength;

(e) measuring the internal reference emission signal at the fourth wavelength;

(f) ratiometricaily determining the pH of the sample using the measurements obtained in steps (c) and (e); and

(g) ratiometricaily determining the oxygen concentration of sample using the measurements obtained in steps (d) and (e).
(72) The method of any of the above (69)-(71), wherein the sample is a single cell or a cell culture.
(73) A method of detecting single cell espiration in a sample comprising cells, wherein the method comprises:

(a) exposing the samp e optical tut inescence dual sensor according to any of the above (1)-(19);

(b) irradiating the sensor at a first wavelength to produce an oxygen indicator emission signal at a second wavelength and an internal reference emission signal at a third wavelength at a first time point;

(c) measuring the oxygen indicator emission signal at the second wavelength;

(d) measuring the internal reference emission signal at the third wavelength;

(e) ratiometrically determining th oxygen concentration in the sample; and

(f) repeating steps (b)-(e) at least at a second time point,

wherein a decrease in the oxygen concentration at the at least second time point indicates cell respiration.
(74) A method of detecting single cell respiration, wherein the method comprises:

(a) exposing a cell to an optical luminescence dual sensor according to any of the above (1)-(19);

(b) irradiating the sensor at a first wavelength to produce an oxygen indicator emission signal at a second wavelength and an internal reference emission signal at a third wavelength at a first time point;

(c) measuring the oxygen indicator emission signal at the second wavelength;

(d) measuring the internal reference emission signal at the third wavelength;

(e) ratiometrically determining the oxygen concentration in the sample; and

(f) repeating steps (b)-(e) at least at a second time point,

wherein a decrease in the oxygen concentration at the at east second time point indicates cell respiration.
(75) A method of determining a cellular respiration rate in a sample, wherein the method comprises:

(a) exposing the sample to an optical luminescence dual sensor according to any of the above (1)-(19);

(b) irradiating the sensor at a first wavelength to produce an oxygen indicator emission signal at a second wavelength and an internal reference emission signal at a third wavelength at a first time point;

(c) measuring the oxygen indicator emission signal at the second wavelength;

(d) measuring the internal reference emission signal at the third wavelength;

(e) ratiometrically determining the oxygen concentration in the sample; and

(f) repeating steps (b)-(e) at least at a second time point; and

(g) determining the respiration rate from the difference in oxygen concentration in the sample at the first time point and the at least second time point as a function of time.
(76) The method of the above (75), wherein the sample is a single cell or a cell culture.
(77) A method of detecting extracellular acidification in a sample, wherein the method comprises:

(a) exposing the sample to an optical luminescence dual sensor according to any of the above (1)-(19);

(b) irradiating the sensor at a first wavelength to produce a pH indicator emission signal at a second wavelength and an internal reference emission signal at a third wavelength at a first time point;

(c) measuring the indicator emission signal at t ie second wavelength;

(d) measuring the internal reference emission signal at the third wavelength;

(e) ratiometrically determining the pH in the sample; and

(f) repeating steps (b)-(e) at least at a second time point,

wherein decrease in the pH at the at least second time point indicates extracellular acidification.
(78) A method of detecting extracellular acidification in a single cell, wherein the method comprises:

(a) exposing the cell to an optical luminescence dual sensor according to any of the above (1)-(19);

(b) irradiating the sensor at a first wavelength to produce a pH indicator emission signal at a second wavelength and an internal reference emission signal at a third wavelength at a first time point;

(c) measuring the pH iodioakxeonixaionainnnl at the second wavelength;

(d) measuring the internal reference emission signal at the third wavelength;

(e) ratiometrically determining the pH in the sample; and

(f) repeating steps (b)-(e) at least at a second time point,

wherein decrease in the pH at the at least second time point indicates extraceilt acidification.
(79) A method of determining extracellular acidification rate in a sample, wherein the method comprises:

(a) exposing the sample to an optical luminescence dual sensor according to any of the above (1)-(19);

(b) irradiating the sensor at a first wavelength to produce a pH indicator emission signal at a second wavelength and an internal reference emission signal at a third wavelength at a first time point;

(c) measuring the pH indicator emission signal at the second wavelength;

(d) measuring the internal reference emission signal at the third wavelength;

(e) ratiometrically determining the the sample;

(f) repeating steps (b)-(e) at least at a second time point; and

(g) determining the extracellular acidification rate from the difference in pH in the sample at the first time point and the at least second time point as a function of time.
(80) A method of simultaneously detecting single cell respiration and extracellular acidification concentration in a sample, wherein the method comprises:

(a) exposing the sample to an optical luminescence dual sensor according to any of the above (1)-(19);

(b) irradiating the sensor (i) at a first wavelength to produce a pH indicator emission signal at a second wavelength, (ii) at a third wavelength to produce an oxygen indicator emission signal at a fourth wavelength and (iii) at a fifth wavelength to produce an internal reference emission signal at a sixth wavelength;

(c) measuring the pH indicator emission signal at the second wavelength;

(d) measuring the oxygen indicator emission signal at the third wavelength;

(e) measuring the internal reference emission signal at the fourth wavelength;

(f) ratiometrically determining the pH of the sample using the measurements obtained in steps (c) and (e);

(g) ratiometricaily determining the oxygen concentration of the sample using the measurements obtained in steps (d) and (e);

(f) repeating steps (b)-(g) at least at a second time point,

wherein a decrease in the oxygen concentration at the at least second time point indicates cell respiration and a decrease in the pH at the at least second time point indicates extracellular acidification.
(81) The method of the above (79) and (80), wherein the sample is a single cell or a cell culture.

Definitions

Unless defined otherwise, all technical and scientific terms used herein have the same meaning as those commonly understood by one of ordinary skill in the art to which this invention belongs. Although methods and materials similar or equivalent to those described herein can be used in the practice or testing of the present disclosure, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

The term “a” or “an” may mean more than one of an item.

The terms “and” and “or” may refer to either the conjunctive or disjunctive and mean “and/or”.

The term “about” means within plus or minus 10% of a stated value. For example, “about 100” would refer to any number between 90 and 110.

The terms “ratiometric method” and “ratiometrically determining” are used interchangeably and are based on the measurement of two probes simultaneously, one that is sensitive to the analyte of interest, and a second that is not, and then taking the ratio of the two [Schaeferling, M., Duerkop, A., 2008. Springer Series on Fluorescence. 5, Springer, 373-414; Xu, H., Aylott, J. W., Kopelman, R., Miller, T. J., Philbert, M. A, 2001, Anal. Chem. 73, 4124-4133; Kermis, H. R., Kostov, V., Harms, P., Rao, G., 2002. Biotechnol. Prog. 18, 1047-1053; Lee, S., Ibey, B., Cote, G. L., Pishko, M. V., 2008. Sens. Actuators B, 128, 388-398.]. The ratiometric method has been known to increase measurement accuracy and to alleviate environmental influences, such as fluctuations in excitation source intensity, variance in probe concentration, and uncontrollable variations in background fluorescence.

The terms “probe for sensing oxygen,” “oxygen probe” and “oxygen sensor” are used interchangeably and may be abbreviated as “OS”.

The terms “pH sensor,” “pH probe” and “probe for sensing pH” are used interchangeably and may be abbreviated as “pHS”.

The term “internal reference probe” may be abbreviated as “IRP”.

The term “polymerized form of a probe” refers to a monomer unit of a probe that is capable of undergoing a polymerization reaction to produce a polymer of the probe or a co-polymer with one or more types of probes or matrices. In a first embodiment, the co-polymer comprises a probe for sensing pH and an internal reference probe. In a second embodiment, the co-polymer comprises a probe for sensing oxygen and an internal reference probe. In a third embodiment, the co-polymer comprises a probe for sensing pH, a probe for sensing oxygen and an internal reference probe. In each of the three embodiments, the co-polymer may further comprise a matrix.

The term “polymerized probe” refers to the polymer product of a probe.

The term “halo” refers to F, Cl, Br, and I.

The term “thermal initiator” refers to a compound that generates a free radical at an elevated temperature.

The term “photoinitiator” refers to a compound that generates a free radical when exposed to light.

The abbreviation “AIBN” refers to 2,2′-azobis(2-methylpropionitrile).

The abbreviation “BPO” refers to benzoyl peroxide.

The term “anion” refers to an ion that is negatively charged. Anions are well-known in the art. In some embodiments, the anion can be halo.

### Sensor Design

The present disclosure provides an optical luminescence dual sensor comprising three probes, each with a different emission color. In particular, the optical luminescence dual sensors comprise a polymerized form of a probe for sensing pH; a polymerized form of a probe for sensing oxygen; a polymerized form of an internal reference probe; and a matrix.

The probe for sensing pH has formula I:

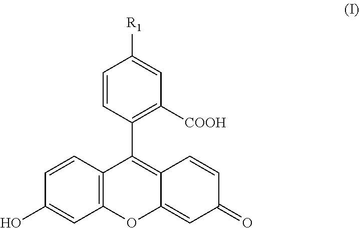

wherein R₁ is CₘH₂ₘX or NHCOCₘH₂ₘY, where m is an integer selected from the group consisting of 0, 1, 2, 3, 4, 5, 6, 8 and 11;

X is selected from the group consisting of:

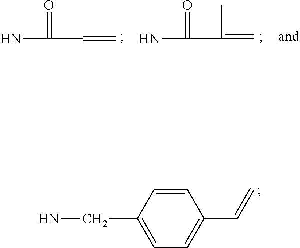

and

Y is selected from the group consisting of:

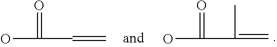

In some embodiments, in the probe for sensing pH, R₁ is CₘH₂ₘX. In other embodiments, in the probe for sensing pH, R₁ is NHCOCₘH₂ₘY.

In some embodiments, in the probe for sensing pH, R₁ is CₘH₂ₘX, where m is 0, 1, 2 or 3. In some embodiments, in the probe for sensing pH, R₁ is CₘH₂ₘX, where m is 0, 1 or 2. In some embodiments, in the probe for sensing pH, R₁ is CₘH₂ₘX, where m is 0, 1 or 3. In some embodiments, in the probe for sensing pH, R₁ is CₘH₂ₘX, where in is 0, 2 or 3. In some embodiments, in the probe for sensing pH, R₁ is CₘH₂ₘX, where m is 0 or 1. In some embodiments, in the probe for sensing pH, R₁ is CₘH₂ₘX, where m is 0 or 2. In some embodiments, in the probe for sensing pH, R₁ is CₘH₂ₘX, where m is 0 or 3. In some embodiments, in the probe for sensing pH, R₁ is CₘH₂ₘX, where m is 1 or 2. In some embodiments, in the probe for sensing pH, R₁ is CₘH₂ₘX, where m is 1 or 3. In some embodiments, in the probe for sensing pH, R₁ is CₘH₂ₘX, where m is 2 or 3. In some embodiments, in the probe for sensing pH, R₁ is CₘH₂ₘX, where m is 0. In some embodiments, in the probe for sensing pH, R₁ is CₘH₂ₘX, where m is 1. In some embodiments, in the probe for sensing pH, R₁ is CₘH₂ₘX, where m is 2. In some embodiments, in the probe for sensing pH, R₁ is CₘH₂ₘX, where m is 3.

In some embodiments, in the probe for sensing pH, X is

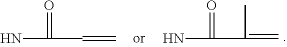

In some embodiments, in the probe for sensing pH, X is

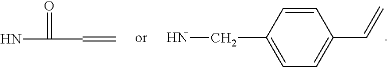

In some embodiments, in the probe for sensing pH, X is

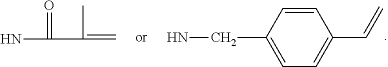

In some embodiments, in the probe for sensing pH, X is

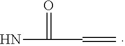

In some embodiments, in the probe for sensing pH. X is

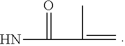

In some embodiments, in the probe for sensing pH, X is

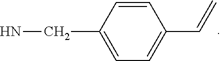

In some embodiments, in the probe for sensing pH, R₁ is CₘH₂ₘX, where m is 0, 1, 2 or 3, and X is

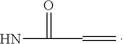

In some embodiments, in the probe for sensing pH, R₁ is CₘH₂ₘX, where m is 0, 1 or 2, and X is

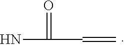

In some embodiments, in the probe for sensing pH, R₁ is CₘH₂ₘX, where m is 0, 1 or 3, and X is

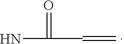

In some embodiments, in the probe for sensing pH, R₁ is CₘH₂ₘX, where m is 0, 2 or 3, and X is

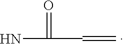

In some embodiments, in the probe for sensing pH, R₁ is CₘH₂ₘX, where m is 0 or 1, and X is

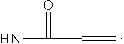

In some embodiments, in the probe for sensing pH, R₁ is CₘH₂ₘX, where m is 0 or 2, and X is

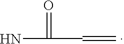

In some embodiments, in the probe for sensing pH, R₁ is CₘH₂ₘX, where m is 0 or 3, and X is

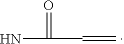

In some embodiments, in the probe for sensing pH, R₁ is CₘH₂ₘX, where m is 1 or 2, and X is

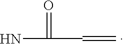

In some embodiments, in the probe for sensing pH, R₁ is CₘH₂ₘX, where m is 1 or 3, and X is

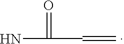

In some embodiments, in the probe for sensing pH, R₁ is CₘH₂ₘX, where m is 2 or 3, and X is

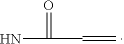

In some embodiments, in the probe for sensing pH, R₁ is CₘH₂ₘX, where m is 0, and X is

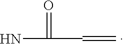

In some embodiments, in the probe for sensing pH, R₁ is CₘH₂ₘX, where m is 1, and X is

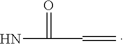

In some embodiments, in the probe for sensing pH, R₁ is CₘH₂ₘX, where m is 2, and X is

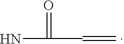

In some embodiments, in the probe for sensing pH, R₁ is CₘH₂ₘX, where m is 3, and X is

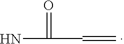

In some embodiments, in the probe for sensing pH, R₁ is CₘH₂ₘX, where m is 0, 1, 2 or 3, and X is

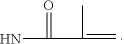

In some embodiments, in the probe for sensing pH, R₁ is CₘH₂ₘX, where m is 0, 1 or 2, and X is

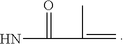

In some embodiments, in the probe for sensing pH, R₁ is CₘH₂ₘX, where m is 0, 1 or 3, and X is

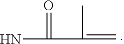

In some embodiments, in the probe for sensing pH, R₁ is CₘH₂ₘX, where m is 0, 2 or 3, and X is

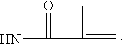

In some embodiments, in the probe for sensing pH, R₁ is CₘH₂ₘX, where m is 0 or 1, and X is

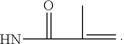

In some embodiments, in the probe for sensing pH, R₁ is CₘH₂ₘX, where m is 0 or 2, and X is

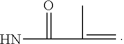

In some embodiments, in the probe tor sensing pH, R₁ is CₘH₂ₘX, where m is 0 or 3, and X is

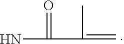

In some embodiments, in the probe for sensing pH, R₁ is CₘH₂ₘX,
where m is 1 or 2, and X is

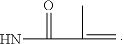

In some embodiments, in the probe for sensing pH, R₁ is CₘH₂ₘX, where m is 1 or 3, and X is

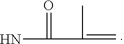

In some embodiments, in the probe for sensing pH, R₁ is CₘH₂ₘX, where m is 2 or 3, and X is

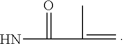

In some embodiments, in the probe for sensing pH, R₁ is CₘH₂ₘX, where m is 0, and X is

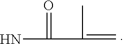

In some embodiments, in the probe for sensing pH, R₁ is CₘH₂ₘX, where m is 1, and X is

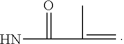

In some embodiments, in the probe for sensing pH, R₁ is CₘH₂ₘX, where m is 2, and X is

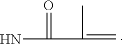

In some embodiments, in the probe for sensing pH, R₁ is CₘH₂ₘX, where m is 3, and X is

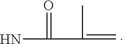

In some embodiments, in the probe for sensing pH, R₁ is CₘH₂ₘX, here m is 0, 1, 2 or 3, and X is

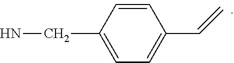

In some embodiments, in the probe for sensing pH, R₁ is CₘH₂ₘX, where m is 0, 1 or 2, and X is

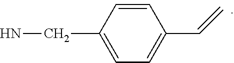

In some embodiments, in the probe for sensing pH, R₁ is CₘH₂ₘX, where m is 0, 1 or 3, and X is

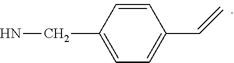

In some embodiments, in the probe for sensing pH, R₁ is CₘH₂ₘX, where m is 0, 2 or 3, and X is

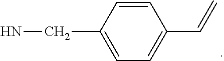

In some embodiments, in the probe for sensing pH, R₁ is CₘH₂ₘX, where m is 0 or 1, and X is

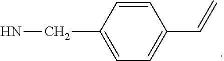

In some embodiments, in the probe for sensing pH, R₁ is CₘH₂ₘX, where m is 0 or 2, and X is

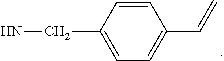

In some embodiments, in the probe for sensing pH, R₁ is CₘH₂ₘX, where m is 0 or 3, and X is

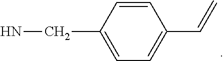

In some embodiments, in the probe for sensing pH, R₁ is CₘH₂ₘX, where m is 1 or 2, and X is

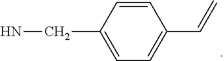

In some embodiments, in the probe for sensing pH, R₁ is CₘH₂ₘX, where m is 1 or 3, and X is

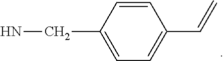

In some embodiments, in the probe for sensing pH, R₁ is CₘH₂ₘX, where m is 2 or 3, and X is

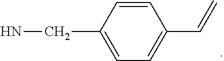

In some embodiments, in the probe for sensing pH, R₁ is CₘH₂ₘX, where m is 0, and X is

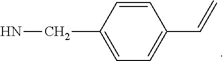

In some embodiments, in the probe for sensing pH, R₁ is CₘH₂ₘX, where m is 1, and X is

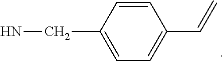

In some embodiments, in the probe for sensing pH, R₁ is CₘH₂ₘX, where m is 2, and X is

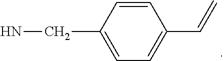

In some embodiments, in the probe for sensing pH, R₁ is CₘH₂ₘX, where m is 3, and X is

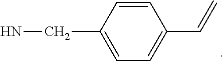

In other embodiments, in the probe for sensing pH, R₁ is NHCOCₘH₂ₘY, where m is 0, 1, 2 or 3. In some embodiments, in the probe for sensing pH, R₁ is NHCOCₘH₂ₘY, where m is 0, 1 or 2. In some embodiments, in the probe for sensing pH, R₁ is NHCOCₘH₂ₘY, where m is 0, 1 or 3. In some embodiments, in the probe for sensing pH, R₁ is NHCOCₘH₂ₘY, where m is 0, 2 or 3. In some embodiments, in the probe for sensing pH, R₁ is NHCOCₘH₂ₘY, where m is 0 or 1. In some embodiments, in the probe for sensing pH, R₁ is NHCOCₘH₂ₘY, where m is 0 or 2. In some embodiments, in the probe for sensing pH, R₁ is NHCOCₘH₂ₘY, where m is 0 or 3. In some embodiments, in the probe for sensing pH, R₁ is NHCOCₘH₂ₘY, where m is 1 or 2. In some embodiments, in the probe for sensing pH, R₁ is NHCOCₘH₂ₘY, where m is 1 or 3. In some embodiments, in the probe for sensing pH, R₁ is NHCOCₘH₂ₘY, where in is 2 or 3. In some embodiments, in the probe for sensing pH, R₁ is NHCOCₘH₂ₘY, where m is 0. In some embodiments, in the probe for sensing pH, R₁ is NHCOCₘH₂ₘY, where in is 1. In some embodiments, in the probe for sensing pH, R₁ is NHCOCₘH₂ₘY, where m is 2. In some embodiments, in the probe for sensing pH, R₁ is NHCOCₘH₂ₘY, where m is 3.

In some embodiments, Y is

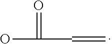

In other embodiments, Y is

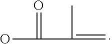

In some embodiments, in the probe for sensing pH, R₁ is NHCOCₘH₂ₘY, where in is 0, 1, 2 or 3, and Y is

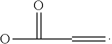

In some embodiments, in the probe for sensing pH, R₁ is NHCOCₘH₂ₘY, where m is 0, 1 or 2, and Y is

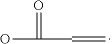

In some embodiments, in the probe for sensing pH, R₁ is NHCOCₘH₂ₘY, where m is 0, 1 or 3, and Y is

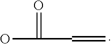

In some embodiments, in the probe for sensing pH, R₁ is NHCOCₘH₂ₘY, where m is 0, 2 or 3, and Y is

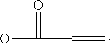

In some embodiments, in the probe for sensing pH, R₁ is NHCOCₘH₂ₘY, where in is 0 or 1, and Y is

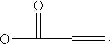

In some embodiments, in the probe for sensing pH, R₁ is NHCOCₘH₂ₘY, where m is 0 or 2, and Y is

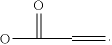

In some embodiments, in the probe for sensing pH, R₁ is NHCOCₘH₂ₘY, where m is 0 or 3, and Y is

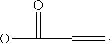

In some embodiments, in the probe for sensing pH, R₁ is NHCOCₘH₂ₘY, where m is 1 or 2, and Y is

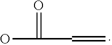

In some embodiments, in the probe tor sensing pH, R₁ is NHCOCₘH₂ₘY, where m is 1 or 3, and Y is

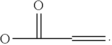

In some embodiments, in the probe for sensing pH, R₁ is NHCOCₘH₂ₘY, where m is 2 or 3, and Y is

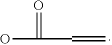

In some embodiments, in the probe for sensing pH, R₁ is NHCOCₘH₂ₘY, where m is 0 and Y is

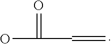

In some embodiments, in the probe for sensing pH, R₁ is NHCOCₘH₂ₘY, where m is 1, and Y is

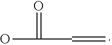

In some embodiments, in the probe for sensing pH, R₁ is NHCOCₘH₂ₘY, where m is 2, and Y is

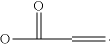

In some embodiments, in the probe for sensing pH, R₁ is NHCOCₘH₂ₘY, where m is 3, and Y is

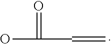

In other embodiments, in the probe for sensing pH, R₁ is NHCOCₘH₂ₘY, where m is 0, 1, 2 or 3, and Y is

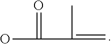

In some embodiments, in the probe for sensing pH, R₁ is NHCOCₘH₂ₘY, where m is 0, 1 or 2, and Y is

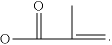

In some embodiments, in the probe for sensing pH, R₁ is NHCOCₘH₂ₘY, where m is 0, 1 or 3, and Y is

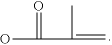

In some embodiments, in the probe for sensing pH, R₁ is NHCOCₘH₂ₘY, where m is 0, 2 or 3, and Y is

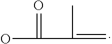

In some embodiments, in the probe for sensing pH, R₁ is NHCOCₘH₂ₘY, where m is 0 or 1, and Y is

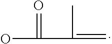

In some embodiments, in the probe for sensing pH, R₁ is NHCOCₘH₂ₘY, where m is 0 or 2, and Y is

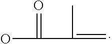

In some embodiments, in the probe for sensing pH, R₁ is NHCOCₘH₂ₘY, where m is 0 or 3, and Y is

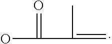

In some embodiments, in the probe for sensing pH, R₁ is NHCOCₘH₂ₘY, where m is 1 or 2, and Y is

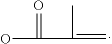

In some embodiments, in the probe for sensing pH, R₁ is NHCOCₘH₂ₘY, where m is 1 or 3, and Y is

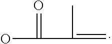

In some embodiments, in the probe for sensing pH, R₁ is NHCOCₘH₂ₘY, where m is 2 or 3, and Y is

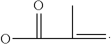

In some embodiments, in the probe for sensing pH, R₁ is NHCOCₘH₂ₘY, where m is 0 and Y is

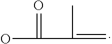

In some embodiments, in the probe for sensing pH, R₁ is NHCOCₘH₂ₘY, where m is 1, and Y is

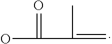

In some embodiments, in the probe for sensing pH, R₁ is NHCOCₘH₂ₘY, where m is 2, and Y is

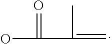

In some embodiments, in the probe for sensing pH, R₁ is NHCOCₘH₂ₘY, where m is 3, and Y is

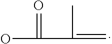

In some embodiments, the probe for sensing pH is:

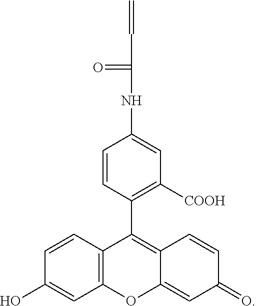 [SMILES: C=CC(=O)Nc1ccc(-c2c3ccc(=O)cc-3oc3cc(O)ccc23)c(C(=O)O)c1]

The probe for sensing oxygen has formula:

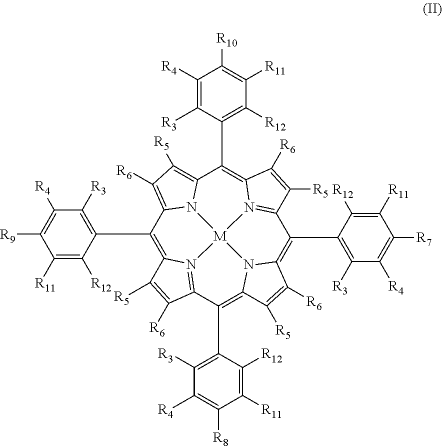

where

M is selected from Pt or Pd;

R₃, R₄, R₅, and R₁₂ can be the same or different and are independently selected from the group consisting of H, halo, CH₃, OCH₃ and OC₂H₅;

R₇, R₈, R₉ and R₁₀ can be the same or different and are independently selected from the group consisting of (CH₂)ₚOH, O(CH₂)ₚOH, NH(CH₂)ₚOH, (CH₂)ₚOM′A, O(CH₂)ₚOM′A, NH(CH₂)ₚOM′A, (CH₂)ₚOA, O(CH₂)ₚOA, NH(CH₂)ₚOA, (CH₂)ₚOVA, O(CH₂)ₚOVA, NH(CH₂)ₚOVA, (OCH₂CH₂)_{q}OH, NH(CH₂CH₂O)_{q}H, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA, NH(CH₂CH₂O)_{q}VA,

where M′A is

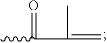

A is

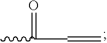

VA is

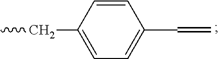

p is an integer selected from the group of consisting of 0, 1, 2, 3, 4, 5, 6, 7, 8, 9, 10, 11 and 12; and

q is an integer selected from the group of consisting of 1, 2, 3, 4, 5, 6, 7, 8, 9, 10, 11, 12, 13, 14, 15, 16, 17, 18, 19, 20, 21, 22, 23, 38, 39, 40, 41, 42, 43, 44, 140, 141, 142, 143, 144, 145, 146, 147, 148, 149 and 150.

In some embodiments, M is Pt. In other embodiments, M is Pd.

In some embodiments, R₁₁ is H, halo, CH₃ or OCH₃. In other embodiments, Ru is H, halo, CH₃ or OC₂H₅. In other embodiments, R₁₁ is H, halo or CH₃. In other embodiments, R₁₁ is H, halo, or OCH₃. In other embodiments, R₁₁ is H, halo, or OC₂H₅. In other embodiments, R₁₁ is halo or H. In other embodiments, R₁₁ is halo. In other embodiments, R₁₁ is H. In other embodiments, R₁₁ is F. In other embodiments, R₁₁ is Cl. In other embodiments, R₁₁ is Br.

In some embodiments, R₁₂ is 14, halo, CH₃ or OCH₃. In other embodiments, R₁₂ is H, halo, CH₃ or OC₂H₅. In other embodiments, R₁₂ is H, halo or CH₃. In other embodiments, R₁₂ is H, halo, or OCH₃. In other embodiments, R₁₂ is H, halo, or OC₂H₅. In other embodiments, R₁₇ is halo or H. In other embodiments, R₁₂ is halo. In other embodiments, R₁₂ is H. In other embodiments, R₁₂ is F. In other embodiments, R₁₂ is Cl. In other embodiments, R₁₂ is Br.

In some embodiments, R₃ is H, halo, CH₃ or OCH₃. In other embodiments, R₃ is H, halo, CH₃ or OC₂H₅. In other embodiments, R₃ is H, halo or CH₃. In other embodiments, R₃ is H, halo, or OCH₃. In other embodiments, R₃ is H, halo, or OC₂H₅. In other embodiments, R₃ is halo or H. In other embodiments, R₃ is halo. In other embodiments, R₃ is H. In other embodiments, R₃ is F. In other embodiments, R₃ is Cl. In other embodiments, R₃ is Br.

In some embodiments, R₄ is H, halo, CH₃ or OCH₃. In other embodiments, R₄ is H, halo, CH₃ or OC₂H₅. In other embodiments, R₄ is H, halo or CH₃. In other embodiments, R₄ is H, halo, or OCH₃. In other embodiments, R₄ is H, halo, or OC₂H₅. In other embodiments, R₄ is halo or H. In other embodiments, R₄ is halo. In other embodiments, R₄ is H. In other embodiments, R₄ is F. In other embodiments, R₄ is Cl. In other embodiments, R₄ is Br.

In some embodiments, R₅ is H, halo, CH₃ or OCH₃. In other embodiments, R₅ is H, halo, CH₃ or OC₂H₅. In other embodiments, R₅ is H, halo or CH₃. In other embodiments, R₅ is H, halo, or OCH₃. In other embodiments, R₅ is H, halo, or OC₂H₅. In other embodiments, R₅ is halo or H. In other embodiments, R₅ is halo. In other embodiments, R₅ is H. In other embodiments, R₅ is F. In other embodiments, R₅ is Cl. In other embodiments, R₅ is Br.

In some embodiments, R₆ is H, halo, CH₃ or OCH₃. In other embodiments, R₆ is H, halo, CH₃ or OC₂H₅. In other embodiments, R₆ is H, halo or CH₃. In other embodiments, R₆ is H, halo, or OCH₃. In other embodiments, R₆ is H, halo, or OC₂H₅. In other embodiments, R₆ is halo or H. In other embodiments, R₆ is halo. In other embodiments, R₆ is H. In other embodiments, R₆ is F. In other embodiments, R₆ is Cl. In other embodiments, R₆ is Br.

In some embodiments, R₇ is O(CH₂)ₚOM′A, NH(CH₂)ₚOM′A, O(CH₂)ₚOA, NH(CH₂)ₚOA, O(CH₂)ₚOVA, NH(CH₂)ₚOVA, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA or NH(CH₂CH₂O)_{q}VA. In some embodiments, R₇ is O(CH₂)ₚOM′A, O(CH₂)ₚOA, O(CH₂)ₚOVA, (OCH₂CH₂)_{q}OM′A, (OCH₂CH₂)_{q}OA or (OCH₂CH₂)_{q}OVA. In some embodiments, R₇ is NH(CH₂)ₚOM′A, NH(CH₂)ₚOA, NH(CH₂)ₚOVA, NH(CH₂CH₂O)_{q}M′A, NH(CH₂CH₂O)_{q}A or NH(CH₂CH₂O)_{q}VA. In some embodiments, R₇ is O(CH₂)ₚOM′A, NH(CH₂)ₚOM′A, (OCH₂CH₂)_{q}OM′A or NH(CH₂CH₂O)_{q}M′A. In some embodiments, R₇ is O(CH₂)ₚOA, NH(CH₂)ₚOA, (OCH₂CH₂)_{q}OA or NH(CH₂CH₂O)_{q}A. In some embodiments, R₇ is O(CH₂)ₚOVA, NH(CH₂)ₚOVA, (OCH₂CH₂)_{q}OVA or NH(CH₂CH₂O)_{q}VA. In some embodiments, R₇ is O(CH₂)ₚOM′A.

In some embodiments, R₇ is O(CH₂)ₚOM′A and p is 1, 2 or 3. In some embodiments, R₇ is O(CH₂)ₚOM′A and p is 1 or 2. In some embodiments, R₇ is O(CH₂)ₚOM′A and p is 1 or 3. In some embodiments, R₇ is O(CH₂)ₚOM′A and p is 2 or 3. In some embodiments, R₇ is O(CH₂)ₚOM′A and p is 1. In some embodiments, R₇ is O(CH₂)ₚOM′A and p is 2. In some embodiments, R₇ iS O(CH₂)ₚOM′A and p is 3.

In some embodiments, R₈ is O(CH₂)ₚOM′A, NH(CH₂)ₚOM′A, O(CH₂)ₚOA, NH(CH₂)ₚOA, O(CH₂)ₚOVA, NH(CH₂)ₚOVA, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA or NH(CH₂CH₂O)_{q}VA. In some embodiments, R₈ is O(CH₂)ₚOM′A, O(CH₂)ₚOA, O(CH₂)ₚOVA, (OCH₂CH₂)_{q}OM′A, (OCH₂CH₂)_{q}OA or (OCH₂CH₂)_{q}OVA. In some embodiments, R₈ is NH(CH₂)ₚOM′A, NH(CH₂)ₚOA, NH(CH₂)ₚOVA, NH(CH₂CH₂O)_{q}M′A, NH(CH₂CH₂O)_{q}A or NH(CH₂CH₂O)_{q}VA. In some embodiments, R₈ is O(CH₂)ₚOM′A, NH(CH₂)ₚOM′A, (OCH₂CH₂)_{q}OM′A or NH(CH₂CH₂O)_{q}M′A. In some embodiments, R₈ is O(CH₂)ₚOA, NH(CH₂)ₚOA, (OCH₂CH₂)_{q}OA or NH(CH₂CH₂O)_{q}A. In some embodiments, R₈ is O(CH₂)ₚOVA, NH(CH₂)ₚOVA, (OCH₂CH₂)_{q}OVA or NH(CH₂CH₂O)_{q}VA. In some embodiments, R₈ is O(CH₂)ₚOM′A.

In some embodiments, R₈ is O(CH₂)ₚOM′A and p is 1, 2 or 3. In some embodiments, R₈ is O(CH₂)ₚOM′A and p is 1 or 2. In some embodiments, R₈ is O(CH₂)ₚOM′A and p is 1 or 3. In some embodiments, R₈ is O(CH₂)ₚOM′A and p is 2 or 3. In some embodiments, R₈ is O(CH₂)ₚOM′A and p is 1. In some embodiments, R₈ is O(CH₂)ₚOM′A and p is 2. In some embodiments, R₈ is O(CH₂)ₚOM′A and p is 3.

In some embodiments, R₉ is O(CH₂)ₚOM′A, NH(CH₂)ₚOM′A, O(CH₂)ₚOA, NH(CH₂)ₚOA, O(CH₂)ₚOVA, NH(CH₂)ₚOVA, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA or NH(CH₂CH₂O)_{q}VA. In some embodiments, R₉ is O(CH₂)ₚOM′A, O(CH₂)ₚOA, O(CH₂)ₚOVA, (OCH₂CH₂)_{q}OM′A, (OCH₂CH₂)_{q}OA or (OCH₂CH₂)_{q}OVA. In some embodiments, R₉ is NH(CH₂)ₚOM′A, NH(CH₂)ₚOA, NH(CH₂)ₚOVA, NH(CH₂CH₂O)_{q}M′A, NH(CH₂CH₂O)_{q}A or NH(CH₂CH₂O)_{q}VA. In some embodiments, R₉ is O(CH₂)ₚOM′A, NH(CH₂)ₚOM′A, (OCH₂CH₂)_{q}OM′A or NH(CH₂CH₂O)_{q}M′A. In some embodiments, R₉ is O(CH₂)ₚOA, NH(CH₂)ₚOA, (OCH₂CH₂)_{q}OA or NH(CH₂CH₂O)_{q}A. In some embodiments, R₉ is O(CH₂)ₚOVA, NH(CH₂)ₚOVA, (OCH₂CH₂)_{q}OVA or NH(CH₂CH₂O)_{q}VA. In some embodiments, R₉ is O(CH₂)ₚOM′A.

In some embodiments, R₉ is O(CH₂)ₚOM′A and p is 1, 2 or 3. In some embodiments, R₉ is O(CH₂)ₚOM′A and p is 1 or 2. In some embodiments, R₉ is O(CH₂)ₚOM′A and p is 1 or 3. In some embodiments, R₉ is O(CH₂)ₚOM′A and p is 2 or 3. In some embodiments, R₉ is O(CH₂)ₚOM′A and p is 1. In sonic embodiments, R₉ is O(CH₂)ₚOM′A and p is 2. In some embodiments, R₉ is O(CH₂)ₚOM′A and p is 3.

In some embodiments, R₁₀ is O(CH₂)ₚOM′A, NH(CH₂)ₚOM′A, O(CH₂)ₚOA, NH(CH₂)ₚOA, O(CH₂)ₚOVA, NH(CH₂)ₚOVA, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA or NH(CH₂CH₂O)_{q}VA. In some embodiments, R₁₀ is O(CH₂)ₚOM′A, O(CH₂)ₚOA, O(CH₂)ₚOVA, (OCH₂CH₂)_{q}OM′A, (OCH₂CH₂)_{q}OA or (OCH₂CH₂)_{q}OVA. In some embodiments, R₁₀ is NH(CH₂)ₚOM′A, NH(CH₂)ₚOA, NH(CH₂)ₚOVA, NH(CH₂CH₂O)_{q}M′A, NH(CH₂CH₂O)_{q}A or NH(CH₂CH₂O)_{q}VA. In some embodiments, R₁₀ is O(CH₂)ₚOM′A, NH(CH₂)ₚOM′A, (OCH₂CH₂)_{q}OM′A or NH(CH₂CH₂O)_{q}M′A. In some embodiments, R₁₀ is O(CH₂)ₚOA, NH(CH₂)ₚOA, (OCH₂CH₂)_{q}OA or NH(CH₂CH₂O)_{q}A. In some embodiments, R₁₀ is O(CH₂)ₚOVA, NH(CH₂)ₚOVA, (OCH₂CH₂)_{q}OVA or NH(CH₂CH₂O)_{q}VA. In some embodiments, R₁₀ is O(CH₂)ₚOM′A.

In some embodiments, R₁₀ is O(CH₂)ₚOM′A and p is 1, 2 or 3. In some embodiments, R₁₀ is O(CH₂)ₚOM′A and p is 1 or 2. In some embodiments, R₁₀ is O(CH₂)ₚOM′A and p is 1 or 3. In some embodiments, R₁₀ is O(CH₂)ₚOM′A and p is 2 or 3. In some embodiments, R₁₀ is O(CH₂)ₚOM′A and p is 1. In some embodiments, R₁₀ is O(CH₂)ₚOM′A and p is 2. In some embodiments, R₁₀ is O(CH₂)OM′A and p is 3.

In some embodiments, M is Pt, and R₃, R₄, R₅, R₆, R₁₁ and R₁₂ are independently halo or H. In some embodiments, M is Pt, and at least one of R₃, R₄, R₅, R₆, R₁₁ and R₁₂ is halo and the other(s) of R₃, R₄, R₅, R₆, R₁₁ and R₁₂ are H. In some embodiments, M is Pt, and at least two of R₃, R₄, R₅, R₆, R₁₁ and R₁₂ are halo and the other(s) of R₃, R₄, R₅, R₆, R₁₁ and R₁₂ are H. In some embodiments, M is Pt, and at least three of R₃, R₄, R₅, R₆, R₁₁ and R₁₂ are halo and the other(s) of R₃, R₄, R₅, R₆, R₁₁ and R₁₂ are H. In some embodiments, M is Pt, and at least four of R₃, R₄, R₅, R₆, R₁₁ and R₁₂ are halo and the other(s) of R₃, R₄, R₅, R₆, R₁₁ and R₁₂ are H. In some embodiments, M is Pt, and at least five of R₃, R₄, R₅, R₆, R₁₁ and R₁₂ are halo and the other of R₃, R₄, R₅, R₆, R₁₁ and R₁₂ is H. In some embodiments, M is Pt, and each of R₃, R₄, R₅, R₆, R₁₁ and R₁₂ is halo.

In some embodiments, M is Pt, and at least one of R₃, R₄, R₅, R₆, R₁₁ and R₁₂ is F and the other(s) of R₃, R₄, R₅, R₆, R₁₁ and R₁₂ are H. In some embodiments, M is Pt, and at least two of R₃, R₄, R₅, R₆, R₁₁ and R₁₂ are F and the other(s) of R₃, R₄, R₅, R₆, R₁₁ and R₁₂ are H. In some embodiments, M is Pt, and at least three of R₃, R₄, R₅, R₆, R₁₁ and R₁₂ are F and the other(s) of R₃, R₄, R₅, R₆, R₁₁ and R₁₂ are H. In some embodiments, M is Pt, and at least tbur of R₃, R₄, R₅, R₆, R₁₁ and R₁₂ are F and the other(s) of R₃, R₄, R₅, R₆, R₁₁ and R₁₂ are H. In some embodiments, M is Pt, and at least five of R₃, R₄, R₅, R₆, R₁₁ and R₁₂ are F and the other of R₃, R₄, R₅, R₆, R₁₁ and R₁₂ is H. In some embodiments, M is Pt, and each of R₃, R₄, R₅, R₆, R₁₁ and R₁₂ is F.

In some embodiments, M is Pt, and at least one of R₃, R₄, R₅, R₆, R₁₁ and R₁₂ is Cl and the other(s) of R₃, R₄, R₅, R₆, R₁₁ and R₁₂ are H. In some embodiments, M is Pt, and at least two of R₃, R₄, R₅, R₆, R₁₁ and R₁₂ are Cl and the other(s) of R₃, R₄, R₅, R₆, R₁₁ and R₁₂ are H. In some embodiments, M is Pt, and at least three of R₃, R₄, R₅, R₆, R₁₁ and R₁₂ are Cl and the other(s) of R₃, R₄, R₅, R₆, R₁₁ and R₁₂ are H. In some embodiments, M is Pt, and at least four of R₃, R₄, R₅, R₆, R₁₁ and R₁₂ are Cl and the other(s) of R₃, R₄, R₅, R₆, R₁₁ and R₁₂ are H. In some embodiments, M is Pt, and at least five of R₃, R₄, R₅, R₆, R₁₁ and R₁₂ are Cl and the other Of R₃, R₄, R₅, R₆, R₁₁ and R₁₂ is H. In some embodiments, M is Pt, and each of R₃, R₄, R₅, R₆, R₁₁ and R₁₂ is Cl.

In some embodiments, M is Pt, R₇ is O(CH₂)ₚOM′A and p is 1, 2 or 3. In some embodiments, M is Pt, R₇ is O(CH₂)ₚOM′A and p is 1 or 2. In some embodiments, M is Pt, R₇ is O(CH₂)ₚOM′A and p is 1 or 3. In some embodiments, M is Pt, R₇ is O(CH₂)ₚOM′A and p is 2 or 3. In some embodiments, M is Pt, R₇ is O(CH₂)ₚOM′A and p is 1. In some embodiments, M is Pt, R₇ is O(CH₂)ₚOM′A and p is 2. In some embodiments, M is Pt, R₇ is O(CH₂)ₚOM′A and p is 3.

In some embodiments, M is Pt, R₈ is O(CH₂)ₚOM′A and p is 1, 2 or 3. In some embodiments, M is Pt, R₈ is O(CH₂)ₚOM′A and p is 1 or 2. In some embodiments, M is Pt, R₈ is O(CH₂)ₚOM′A and p is 1 or 3. In some embodiments, M is Pt, R₈ is O(CH₂)ₚOM′A and p is 2 or 3. In some embodiments, M is Pt, R₈ is O(CH₂)ₚOM′A and p is 1. In some embodiments, M is Pt, R₈ is O(CH₂)ₚOM′A and p is 2. In some embodiments, M is Pt, R₈ is O(CH₂)ₚOM′A and p is 3.

In some embodiments, M is Pt, R₉ is O(CH₂)ₚOM′A and p is 1, 2 or 3. In some embodiments, M is Pt, R₉ is O(CH₂)ₚOM′A and p is 1 or 2. In some embodiments, M is Pt, R₉ is O(CH₂)ₚOM′A and p is 1 or 3. In some embodiments, M is Pt, R₉ is O(CH₂)ₚOM′A and p is 2 or 3. In some embodiments, M is Pt, R₉ is O(CH₂)ₚOM′A and p is 1. In some embodiments, M is Pt, R₉ is O(CH₂)ₚOM′A and p is 2. In some embodiments, M is Pt, R₉ is O(CH₂)ₚOM′A and p is 3.

In some embodiments, M is Pt, R₁₀ is O(CH₂)ₚOM′A and p is 1, 2 or 3. In some embodiments, M is Pt, R₁₀ is O(CH₂)ₚOM′A and p is 1 or 2. In some embodiments, M is Pt, R₁₀ is O(CH₂)ₚOM′A and p is 1 or 3. In some embodiments, M is Pt, R₁₀ is O(CH₂)ₚOM′A and p is 2 or 3. In some embodiments, M is Pt, R₁₀ is O(CH₂)ₚOM′A and p is 1. In some embodiments, M is Pt, R₁₀ is O(CH₂)ₚOM′A and p is 2. In some embodiments, M is Pt, R₁₀ is O(CH₂)ₚOM′A and p is 3.

In some embodiments, the probe for sensing oxygen is:

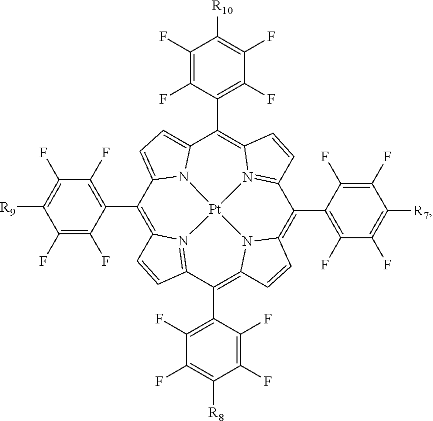

wherein R₇, R₈, R₉ and R₁₀ are

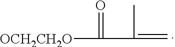

The internal reference probe has formula

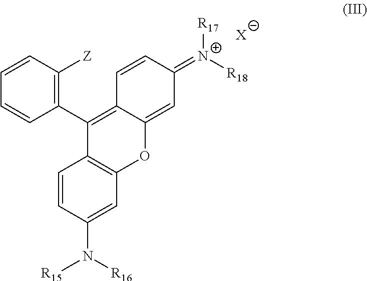

wherein R₁₅, R₁₆, R₁₇, and R₁₈ can be the same or different and are independently CₙH₂ₙ₊₁, where ti is an integer selected from the group consisting of 1, 2, 3, 4, 5, 6, 7 and 8;

X is an anion;

Z is selected from the group consisting of: (CH₂)ₚOH, O(CH₂)ₚOH, NH(CH₂)ₚOH, (CH₂)ₚOM′A, O(CH₂)ₚOM′A, NH(CH₂)ₚOM′A, (CH₂)ₚOA, O(CH₂)ₚOA, NH(CH₂)ₚOA, (CH₂)ₚOVA, O(CH₂)ₚOVA, NH(CH₂)ₚOVA, (OCH₂CH₂)_{q}OH, NH(CH₂CH₂O)_{q}H, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA, NH(CH₂CH₂O)_{q}VA, CH₂(OCH₂CH₂)ᵣOA, CH₂(OCH₂CH₂)ᵣOM′A, CH₂(OCH₂CH₂)ᵣOVA;

p is an integer selected fro oup of consisting of 0, 1, 2, 3, 4, 5, 6, 7, 8, 9, 10, 11 and 12;

q is an integer selected from the group of consisting of 1, 2, 3, 4, 5, 6, 7, 8, 9, 10, 11, 12, 13, 14, 15, 16, 17, 18, 19, 20, 21, 22, 23, 38, 39, 40, 41, 42, 43, 44, 140, 141, 142, 143, 144, 145, 146, 147, 148, 149 and 150; and

r is an integer selected from the group of consisting of 1, 2, 3, 4, 5, 6, 7, 8, 9, 10, 11, 12, 13, 14, 15, 16, 17, 18, 19, 20, 21, 22, 23, 38, 39, 40, 41, 42, 43, 44, 140, 141, 142, 143, 144, 145, 146, 147, 148, 149 and 150.

In some embodiments, in the internal reference probe, R₁₅, R₁₆, R₁₇, R₁₈ are independently CₙH₂ₙ₊₁, where n is an integer selected from the group consisting of 1, 2, 3, 4, 5 and 6. In some embodiments, in the internal reference probe, R₁₅, R₁₆, R₁₇, R₁₈ are independently CₙH₂ₙ₊₁, where n is an integer selected from the group consisting of 1, 2 and 3. In some embodiments, in the internal reference probe, R₁₅, R₁₆, R₁₇, R₁₈ are independently CₙH₂ₙ₊₁, where n is 1. In some embodiments, in the internal reference probe, R₁₅, R₁₆, R₁₇, R₁₈ are independently CₙH₂ₙ₊₁, where n is 2. In some embodiments, in the internal reference probe, R₁₅, R₁₆, R₁₇, R₁₈ are independently CₙH₂ₙ₊₁, where n is 3.

In some embodiments, in the internal reference probe, X is halo. In some embodiments, in the internal reference probe, X is Cl. In some embodiments, in the internal reference probe, X is F. In some embodiments, in the internal reference probe, X is Br.

In some embodiments, in the internal reference probe, Z is (CH₂)ₚOH, (CH₂)ₚOM′A, (CH₂)ₚOA, (CH₂)ₚOVA, CH₂(OCH₂CH₂)ᵣOA, CH₂(OCH₂CH₂)ᵣOM′A or CH₂(OCH₂CH₂)ᵣOVA. In some embodiments, Z is (CH₂)ₚOM′A or CH₂(OCH₂CH₂)ᵣOM′A. In some embodiments, Z is (CH₂)ₚOA, or CH₂(OCH₂CH₂)ᵣOA. In some embodiments, Z is (CH₂)ₚOVA or CH₂(OCH₂CH₂)ᵣOVA. In some embodiments, Z is (CH₂)ₚOM′A.

In some embodiments, Z is (CH₂)ₚOM′A and p is 1, 2 or 3. In some embodiments, Z is (CH₂)ₚOM′A and p is 1 or 2. In some embodiments, Z is (CH₂)ₚOM′A and p is 1 or 3. In some embodiments, Z is (CH₂)ₚOM′A and p is 2 or 3. In some embodiments, Z is (CH₂)ₚOM′A and p is 1. In some embodiments, Z is (CH₂)ₚOM′A and p is 2. In some embodiments, Z is (CH₂)ₚOM′A and p is 3.

In some embodiments, at least one of R₁₅, R₁₆, R₁₇ and R₁₈ is CₙH₂ₙ₊₁, where n is 2. In some embodiments, at least two of R₁₅, R₁₆, R₁₇ and R₁₈ is CₙH₂ₙ₊₁, where n is 2. In some embodiments, at least three of R₁₅, R₁₆, R₁₇ and R₁₈ is CₙH₂ₙ₊₁, where n is 2. In some embodiments, each of R₁₅, R₁₆, R₁₇, R₁₈ is CₙH₂ₙ₊₁, where n is 2.

In some embodiments, X is halo and at least one of R₁₅, R₁₆, R₁₇ and R₁₈ is CₙH₂ₙ₊₁, where n is 2. In some embodiments, X is halo and at least two of R₁₅, R₁₆, R₁₇ and R₁₈ is CₙH₂ₙ₊₁, where n is 2. In some embodiments, X is halo and at least three of R₁₅, R₁₆, R₁₇ and R₁₈ is C₂H₂ₙ₊₁ where n is 2. In some embodiments, X is halo and each of R₁₅, R₁₆, R₁₇, R₁₈ is CₙH₂ₙ₊₁, where n is 2.

In some embodiments. X is chloro and at least one of R₁₅, R₁₆, R₁₇ and R₁₈ is CₙH₂₊₁, where n is 2. In some embodiments, X is chloro and at least two of R₁₅, R₁₆, R₁₇ and R₁₈ is CₙH₂ₙ₊₁, where n is 2. In some embodiments, X is chloro and at least three of R₁₅, R₁₆, R₁₇ and R₁₈ is CₙH₂ₙ₊₁, where n is 2. In some embodiments, X is chloro and each of R₁₅, R₁₆, R₁₇, R₁₈ is CₙH₂ₙ₊₁, where n is 2.

In some embodiments, X is fluoro and at least one of R₁₅, R₁₆, R₁₇ and R₁₈ is CₙH₂ₙ₊₁, where n is 2. In some embodiments, X is fluoro and at least two of R₁₅, R₁₆, R₁₇ and R₁₈ is CₙH₂ₙ₊₁, where n is 2. In some embodiments, X is fluoro and at least three of R₁₅, R₁₆, R₁₇ and Ris is CₙH₂ₙ₊₁, where n is 2. in some embodiments, X is fluoro and each of R₁₅, R₁₆, R₁₇, R₁₈ is CₙH₂ₙ₊₁, where n is 2.

In some embodiments, Z is (CH₂)ₚOM′A, p is 1, 2 or 3 and at least one of R₁₅, R₁₆, R₁₇ and R₁₈ is CₙH₂ₙ₊₁, where n is 2. In some embodiments, Z is (CH₂)ₚOM′A, p is 1 or 2 and at least one of R₁₅, R₁₆, R₁₇ and R₁₈ is CₙH₂ₙ₊₁, where n is 2. In some embodiments, Z is (CH₂)ₚOM′A, p is 1 or 3 and at least one of R₁₅, R₁₆, R₁₇ and R₁₈ is CₙH₂ₙ₊₁, where n is 2. In some embodiments, Z is (CH₂)ₚOM′A, p is 2 or 3 and at least one of R₁₅, R₁₆, R₁₇ and R₁₈ is CₙH₂ₙ₊₁, where n is 2. In some embodiments, Z is (CH₂)ₚOM′A, p is 1 and at least one of R₁₅, R₁₆, R₁₇ and R₁₈ is CₙH₂ₙ₊₁, where n is 2. In some embodiments, Z is (CH₂)ₚOM′A, p is 2 and at least one of R₁₅, R₁₆, R₁₇ and R₁₈ is CₙH₂ₙ₊₁, where n is 2. In some embodiments, Z is (CH₂)ₚOM′A, p is 3 and at least one of R₁₅, R₁₆, R₁₇ and R₁₈ is CₙH₂ₙ₊₁, where n is 2.

In some embodiments, Z is (CH₂)ₚOM′A, p is 1, 2 or 3 and each of R₁₅, R₁₆, R₁₇ and R₁₈ is CₙH₂ₙ₊₁, where n is 2. In some embodiments, Z is (CH₂)ₚOM′A, p is 1 or 2 and each of R₁₅, R₁₆, R₁₇ and R₁₈ is CₙH₂ₙ₊₁, where n is 2. In some embodiments, Z is (CH₂)ₚOM′A, p is 1 or 3 and each of R₁₅, R₁₆, R₁₇ and R₁₈ is CₙH₂ₙ₊₁, where n is 2. In some embodiments, Z is (CH₂)ₚOM′A, p is 2 or 3 and each of R₁₅, R₁₆, R₁₇ and R₁₈ is CₙH₂ₙ₊₁, where n is 2. In some embodiments, Z is (CH₂)ₚOM′A, p is 1 and each of R₁₅, R₁₆, R₁₇ and R₁₈ is CₙH₂₊₁, where n is 2. In some embodiments, Z is (CH₂)ₚOM′A, p is 2 and each of R₁₅, R₁₆, R₁₇ and R₁₈ is CₙH₂ₙ₊₁, where n is 2. In some embodiments, Z is (CH₂)ₚOM′A, p is 3 and each of R₁₅, R₁₆, R₁₇ and R₁₈ is CₙH₂ₙ₊₁, where n is 2

In some embodiments, X is halo, Z is (CH₂)ₚOM′A and p is 1, 2 or 3. In some embodiments, X is halo, Z is (CH₂)ₚOM′A and p is 1 or 2. In some embodiments, X is halo, Z is (CH₂)ₚOM′A and p is 1 or 3. In some embodiments, X is halo, Z is (CH₂)ₚOM′A and p is 2 or 3. In some embodiments, X is halo, Z is (CH₂)ₚOM′A and p is 1. In some embodiments, X is halo, Z is (CH₂)ₚOM′A and p is 2. In some embodiments, X is halo, Z is (CH₂)ₚOM′A and p is 3.

In some embodiments, X is chloro, Z is (CH₂)ₚOM′A and p is 1, 2 or 3. In some embodiments, X is chloro, Z is (CH₂)ₚOM′A and p is 1 or 2. In some embodiments, X is chloro, Z is (CH₂)ₚOM′A and p is 1 or 3. In some embodiments, X is chloro, Z is (CH₂)ₚOM′A and p is 2 or 3. In some embodiments, X is chloro, Z is (CH₂)ₚOM′A and p is 1. In some embodiments, X is chloro, Z is (CH₂)ₚOM′A and p is 2. In some embodiments, X is halo, Z is (CH₂)ₚOM′A and p is 3.

In some embodiments, X is fluoro, Z is (CH₂)ₚOM′A and p is 1, 2 or 3. In some embodiments, X is fluoro, Z is (CH₂)ₚOM′A and p is 1 or 2. In some embodiments, X is fluoro, Z is (CH₂)ₚOM′A and p is 1 or 3. In some embodiments, X is fluoro, Z is (CH₂)ₚOM′A and p is 2 or 3. In some embodiments. X is fluoro, Z is (CH₂)ₚOM′A and p is 1. In some embodiments, X is fluoro, Z is (CH₂)ₚOM′A and p is 2. In some embodiments, X is fluoro, Z is (CH₂)ₚOM′A and p is 3.

In some embodiments, the internal reference probe is:

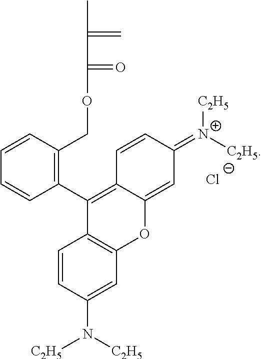 [SMILES: C=C(C)C(=O)OCc1ccccc1-c1c2ccc(=[N+](CC)CC)cc-2oc2cc(N(CC)CC)ccc12 ; Cl]

In some embodiments, the matrix comprises poly(2-hydroxylethyl methacrylate)-co-polyacrylamide (PHEMA-co-PAM).

In some embodiments, the matrix comprises copolymers selected from the group consisting of PHEMA-co-PAM, POEGMA-co-PAM, PNIPAAm-co-PREMA, PNIPAAm-co-PAM and POEGMA-co-PHEMA.

In some embodiments, the optical luminescence dual sensor further comprises a substrate.

In embodiments in which the optical luminescence dual sensor comprises a substrate, the substrate may be selected from the group consisting of quartz glass, fused silica, silica particles, silica gels, and poly(ethylene terephthalate). In some embodiments, the substrate is quartz glass. In other embodiments, the substrate is fused silica. In other embodiments, the substrate is poly(ethylene terephthalate).

In some embodiments, the copolymer is immobilized or attached to the substrate. In some aspects of this embodiment, the copolymer is attached to the substrate using a conjugating layer.

In some embodiments in which the copolymer is attached to the substrate using a conjugating layer, the conjugating layer comprises a silane. In some aspects of this embodiment, the silane is a trimethoxysilane. In some aspects, the silane is 3-acryloxypropyl trimethoxysilane.

In some embodiments, the copolymer is photopatterned on the substrate.

The pH probe, the O₂ probe, and the internal reference probe each have a different emission color. In some embodiments, the three optical probes have well separated spectral windows. In some embodiments, the three optical probes can he excited using the same excitation wavelength.

### Methods of Preparing the Sensors

The present disclosure provides a method of preparing an optical luminescence dual sensor by photo-polymerization. The method comprises copolymerizing a probe for sensing pH, a probe for sensing oxygen, and an internal reference probe, with polyacrylamide, and poly(2-hydroxyethyl methacrylate)-co-poiyacryiamide (PHEMA-co-PAM) in the presence of a crossiinker and an initiator. The probe for sensing pH, the probe for sensing oxygen and the internal reference probe can be any of the probes described above.

Suitable crossiinkers that can be used in the first step of the method include, but are not limited to, SR454, poly(ethylene glycol) diacrylate and poly(ethylene glycol) dimethacrylate. In some aspects, the crosslinker is SR454.

The initiator used in the first step of the method can be a photo-initiator or a thermal initiator. Suitable photo-initiators that can be used include, but are not limited to, IRACURE® 819, 4-phenyl benzophenone, methyl o-benzoyl benzoate and benzyl dimethyl ketal. In some aspects, the photo-initiator is IRACURE® 819. Suitable thermal initiators that can be used include, but are not limited to AIBN and BPO.

In some embodiments, the copolymerization step comprises exposing a mixture comprising a probe for sensing pH, a probe for sensing oxygen, an internal reference probe, matrix, and a photo-initiator under suitable wavelengths, such as, 405 nm and 435 nm, for a certain time (5 to 150 seconds with an interval of 5 seconds).

Next, the copolymer is immobilized or attached onto a substrate.

In some embodiments, the copolymer is attached to the substrate using a conjugating layer. In some aspects, the conjugating layer comprises a silane, such as, a trimethoxysilane. In some aspects, the silane is 3-acryloxypropyl trimethoxysilane.

In some embodiments, the substrate is selected from the group consisting of quartz glass, fused silica, silica particles, silica gels, and polyethylene terephthalate). In some aspects, the substrate is quartz glass. In other aspects, the substrate is fused silica. In other aspects, the substrate is polyethylene terephthalate).

The present disclosure also provides a method of preparing a dual pH and oxygen array on a substrate. The method comprises (a) masking the substrate to define boundaries on the substrate; (b) contacting the unmasked substrate with a conjugate compound to form a conjugated layer-substrate; (c) contacting the conjugated layer-substrate with the sensor as defined above.

In some embodiments, the conjugating layer comprises a silane, such as, a trimethoxysilane. In some aspects, the silane is 3-acryloxypropyl trimethoxysilane.

In some embodiments, the substrate is selected from the group consisting of quartz glass, fused silica, silica particles, silica gels, and poly(ethylene terephthalate). In some aspects, the substrate is quartz glass. In other aspects, the substrate is fused silica. In other aspects, the substrate is polyethylene terephthalate).

In some embodiments, step (c) comprises polymerizing: (i) a probe for sensing pH; (ii) a probe for sensing oxygen; (iii) an internal reference probe; and (iv) a matrix. In some aspects, the polymerization step is in the presence of a photo-initiator.

The present disclosure also provides a dual pH and oxygen sensor pattern produced by the method of preparing a dual pH and oxygen array on a substrate. In some embodiments, the sensor pattern is a photo-pattern of tri-color dual sensors. The sensor pattern can have a photo-pattern of the tri-color dual sensor in any pattern and size. For example, in some embodiments, the sensor pattern is a photo-pattern of any pattern from 2×2 arrays through 1000 s×1000 s arrays. In some embodiments, the sensor pattern is a photo-pattern of 2×2 arrays, 3×3 arrays, 4×4 arrays, 5×5 arrays, 6×6 arrays, 7×7 array, 8×8 arrays, 9×9 arrays and 10×10 arrays. In some embodiments, the sensor pattern is a photo-pattern of 2×2 arrays, 3×3 arrays, 4×4 arrays and 5×5 arrays. In some embodiments, the sensor pattern is a photo-pattern of 2×2 arrays and 3×3 arrays. In some embodiments, the sensor pattern is a photo-pattern of 2×2 arrays. In some embodiments, the sensor pattern is a photo-pattern of 3×3 arrays.

In some embodiments, the sensor pattern is a photo-pattern of tri-color dual sensors having a diameter from about 1 μm through about 200 μm. In some embodiments, the sensor pattern is a photo-pattern of tri-color dual sensors having a diameter from about 1 μm through about 100 μm. In some embodiments, the sensor pattern is a photo-pattern of tri-color dual sensors having a diameter from about 1 μm through about 85 μm. In some embodiments, the sensor pattern is a photo-pattern of tri-eolor dual sensors having a diameter from about 1 μm through about 75 μm. In some embodiments, the sensor pattern is a photo-pattern of tri-color dual sensors having a diameter from about 1 μm through about 60 μm. In some embodiments, the sensor pattern is a photo-pattern of tri-color dual sensors having a diameter from about 10 μm through about 100 μm. In some embodiments, the sensor pattern is a photo-pattern of tri-color dual sensors having a diameter from about 25 um through about 100 μm. In some embodiments, the sensor pattern is a photo-pattern of tri-color dual sensors having a diameter from about 40 μm through about 100 μm. In some embodiments, the sensor pattern is a photo-pattern of tri-color dual sensors having a diameter from about 10 μm through about 85 μm. In some embodiments, the sensor pattern is a photo-pattern of tri-color dual sensors having a diameter from about 25 μm through about 75 μm. In some embodiments, the sensor pattern is a photo-pattern of tri-color dual sensors having a diameter from about 40 μm through about 60 μm. In some embodiments, the sensor pattern is a photo-pattern of tri-color dual sensors having a diameter of about 50 μm. In each embodiment described in this paragraph, the sensor pattern can have a photo-pattern of the tri-color dual sensor in any size. For example, in one embodiment, the sensor pattern is a photo-pattern of any pattern from 2×2 arrays through 1000 s×1000 s arrays. In a second embodiment, the sensor pattern is a photo-pattern of 2×2 arrays, 3×3 arrays, 4×4 arrays, 5×5 arrays, 6×6 arrays, 7×7 array, 8×8 arrays, 9×9 arrays and 10×10 arrays. In a third embodiment, the sensor pattern is a photo-pattern of 2×2 arrays, 3×3 arrays, 4×4 arrays and 5×5 arrays. In a fourth embodiment, the sensor pattern is a photo-pattern of 2×2 arrays and 3×3 arrays. In a fifth embodiment, the sensor pattern is a photo-pattern of 2×2 arrays. In a sixth embodiment, the array is a photo-pattern of 3×3 arrays.

### Methods of Using the Sensors

The present disclosure provides a method of determining the pH of a sample. The method comprises exposing a sample to an optical luminescence dual sensor. The optical luminescence dual sensor can be any of the sensors described above.

The sensor is then irradiated at a first wavelength to produce a pH indicator emission signal at a second wavelength and an internal reference emission signal at a third wavelength. The pH indicator emission signal is measured at the second wavelength and the internal reference emission signal is measured at the third emission wavelength. The pH of the sample is then determined ratiometrically.

In some embodiments, the first wavelength is in the range of about 360 nm to about 500 nm. In some embodiments, the first wavelength is in the range of about 420 nm to about 500 nm. In some embodiments, the first wavelength is about 380 nm. In some embodiments, the first wavelength is about 470 nm.

In some embodiments, the second wavelength is in the range of 490 nm to about 550 nm. some embodiments, the second wavelength is about 521 nm.

In some embodiments, the third wavelength is in the range of about 390 nm to about 630 nm. In some embodiments, the third wavelength is in the range of about 575 nm to about 630 nm. In some embodiments, the third wavelength is in the range of about 390 nm to about 450 nm. In some embodiments, the third wavelength is about 421 nm. In some embodiments, the third wavelength is about 575 nm.

The present disclosure also provides a method of determining the concentration of oxygen in a sample. The method comprises exposing the sample to an optical luminescence dual sensor. The optical luminescence dual sensor can be any of the sensors described above.

The sensor is then irradiated at a first wavelength to produce an oxygen indicator emission signal at a second wavelength and an internal reference emission signal at a third wavelength. The oxygen indicator emission signal is measured at the second wavelength and the internal reference emission signal is measured at the third wavelength. The oxygen concentration in the sample is then determined ratiometrically.

In some emb diments, the first wavelength is in the range of about 360 nm to about 500 nm. In some embodiments, the first wavelength is in the range of about 420 nm to about 500 nm. In some embodiments, the first wavelength is about 380 nm. In some eMbodiments, the first wavelength is about 470 nm.

In some embodiments, the second wavelength is in the range of about 620 nm to about 680 nm. In some embodiments, the second wavelength is about 650 nm.

In some embodiments, the third wavelength is in the range of about 390 nm to about 630 nm. In some embodiments, the third wavelength is in the range of about 575 nm to about 630 nm. In some embodiments, the third wavelength is in the range of about 390 nm to about 450 nm. In some embodiments, the third wavelength is about 421 nm. In some embodiments, the third wavelength is about 575 nm.

The present disclosure additionally provides a method of simultaneously determining the pH and oxygen concentration in a sample. The method comprises exposing the sample to an optical luminescence dual sensor. The optical luminescence dual sensor can be any of the sensors described above.

The sensor is irradiated (i) at a first wavelength to produce a pH indicator emission signal at a. second wavelength; (ii) at a third wavelength to produce an oxygen indicator emission signal at a fourth wavelength and (iii) at a fifth wavelen h to produce an internal reference emission signal at a sixth wavelength. The pH indicator emission signal is measured at the second wavelength, the oxygen indicator emission signal is measured at the fourth wavelength and the internal reference emission signal is measured at the sixth wavelength. The pH of the sample is then determined ratiometrically using the measurements obtained at the second and sixth wavelengths; and the oxygen concentration of the sample is determined ratiometrically using the measurements obtained at the fourth and sixth wavelengths.

In some emb diments, the first wavelength is in the range of about 360 nm to about 500 nm. In some embodiments, the first wavelength is in the range of about 420 nm to about 500 nm. In some embodiments, the first wavelength is about 380 nm. In some embodiments, the first wavelength is about 470 nm.

In some embodiments, the second wavelength is in the range of about 490 nm to about 550 nm. In some embodiments, the second wavelength is about 521 nm.

In some embodiments, the third wavelength is in the range of about 360 nm to about 500 nm. In some embodiments, the third wavelength is in the range of about 420 nm to about 500 nm. In some embodiments, the third wavelength is about 380 nm. In some embodiments, the third wavelength is about 470 nm.

In some embodiments, the fourth wavelength is in the range of about 620 nm to about 680 nm. In some embodiments, the third wavelength is about 650 nm.

In some embodiments, the fifth wavelength is in the range of about 450 nm to about 520 nm. In some embodiments, the fourth wavelength is about 490 nm.

In some embodiments, the sixth wavelength is in the range of about 390 nm to about 630 nm. In some embodiments, the sixth wavelength is in the range of about 575 mn to about 630 nm. In some embodiments, the sixth wavelength is in the range of about 390 nm to about 450 nm. In some embodiments, the sixth wavelength is about 421 nm. In some embodiments, the third wavelength is about 575 nm.

In each of the methods described above, more than one sample can be used. Thus, the method can be performed in a high throughput format.

In each of the methods described above, the sample is a single cell or can be obtained from a cell culture, blood, urine, tear, industry fermentor, photobioreactor, pond, river, lake or ocean.

The methods described herein can be used to monitor, measure or detect cell respiration. In some embodiments, the method is used to monitor, measure or detect cell respiration in a sample. In other embodiments, the method is used to monitor, measure or detect cell respiration in a single cell.

In some embodiments, the method is used to detect cell respiration in a sample. The method comprises: (a) exposing the sample to an optical luminescence dual sensor as defined above; (b) irradiating the sensor at a first wavelength to produce an oxygen indicator emission signal at a second wavelength and an internal reference emission signal at a third wavelength at a first time point; (c) measuring the oxygen indicator emission signal at the second wavelength; (d) measuring the internal reference emission signal at the third wavelength; (e) ratiometrically determining the oxygen concentration in the sample; and (f) repeating steps (b)-(e) at least at a second time point. A decrease in the oxygen concentration at the at least second time point indicates cell respiration.

In some embodiments, the method is used to detect single cell respiration. The method comprises: (a) exposing the cell to an optical luminescence dual sensor as defined above; (b) irradiating the sensor at a first wavelength to produce an oxygen indicator emission signal at a second wavelength and an internal reference emission signal at a third wavelength at a first time point; (c) measuring the oxygen indicator emission signal at the second wavelength; (d) measuring the internal reference emission signal at the third wavelength; (e) ratiometrically determining the oxygen concentration in the sample; and (f) repeating steps (b)-(e) at least at a second time point. A decrease in the oxygen concentration at the at least second time point indicates cell respiration.

The methods described herein can be used to determine cell respiration rate. In some embodiments, the method is used to determine cell respiration rate in a sample comprising cells. In other embodiments, the method is used to determine cell respiration rate in a single cellin some embodiments, the method is used to determine cell respiration rate in a sample comprising cells. The method comprises: (a) exposing the sample to an optical luminescence dual sensor as defined above; (b) irradiating the sensor at a first wavelength to produce an oxygen indicator emission signal at a second wavelength and an internal reference emission signal at a third wavelength at a first time point; (c) measuring the oxygen indicator emission signal at the second wavelength; (d) measuring the internal reference emission signal at the third wavelength; (e) ratiometrically determining the oxygen concentration in the sample; (f) repeating steps (b)-(e) at least at a second time point; and (g) determining the cell respiration rate from the difference in oxygen concentration in the sample at the first time point and the at least second time point as a function of time.

In some embodiments, the method is used to determine single cell respiration rate. The method comprises: (a) exposing the cell to an optical luminescence dual sensor as defined above; (b) irradiating the sensor at a first wavelength to produce an oxygen indicator emission signal at a second wavelength and an internal reference emission signal at a third wavelength at a first time point; (c) measuring the oxygen indicator emission signal at the second wavelength; (d) measuring the internal reference emission signal at the third wavelength; (e) ratiometrically determining the oxygen concentration in the sample; and (f) repeating steps (b)-(e) at least at a second time point.

The methods described herein can be used to monitor, measure or detect extracellular acidification. In some embodiments, the method is used to detect extracellular acidification in a sample. In other embodiments, the method is used to detect extracellular acidification in a single cell.

In some embodiments, the method is used to detect extracellular acidification in a sample. The method comprises: (a) exposing the sample to an optical luminescence dual sensor as defined above; (b) irradiating the sensor at a first wavelength to produce a pH indicator emission signal at a second wavelength and an internal reference emission signal at a third wavelength at a first time point; (c) measuring the pH indicator emission signal at the second wavelength; (d) measuring the internal reference emission signal at the third wavelength; (e) ratiometrically determining the pH in the sample; and (f) repeating steps (b)-(e) at least at a second time point. A decrease in the pH at the at least second time point indicates extracellular acidification.

In some embodiments, the method is used to detect extracellular acidification in a single cell. The method comprises: (a) exposing the sample to an optical luminescence dual sensor as defined above; (b) irradiating the sensor at a first wavelength to produce a pH indicator emission signal at a second wavelength and an internal reference emission signal at a third wavelength at a first time point; (c) measuring the pH indicator emission signal at the second wavelength; (d) measuring the internal reference emission signal at the third wavelength; (e) ratiometrically determining the pH in the sample; and (f) repeating steps (b)-(e) at least at a second time point. A decrease in the pH at the at least second time point indicates extracellular acidification.

The methods described herein can be used to determine extracellular acidification rate. In some embodiments, the method is used to determine the extracellular acidification rate in a sample comprising cells. In other embodiments, the method is used to determine the extracellular acidification rate in a single cell.

In some embodiments, the method is used to determine extracellular acidification rate in a sample. The method comprises: (a) exposing the sample to an optical luminescence dual sensor as defined above; (b) irradiating the sensor at a first wavelength to produce a pH indicator emission signal at a second wavelength and an internal reference emission signal at a third wavelength at a first time point; (c) measuring the pH indicator emission signal at the second wavelength; (d) measuring the internal reference emission signal at the third wavelength; (e) ratiometrically determining the pH in the sample; (f) repeating steps (b)-(e) at least at a second time point; and (g) determining the extracellular acidification rate from the difference in pH in the sample at the first time point and the at least second time point as a function of time.

In some embodiments, the method is used to determine extracellular acidification rate in a single cell. The method comprises: (a) exposing the cell to an optical luminescence dual sensor as defined above; (b) irradiating the sensor at a first wavelength to produce a pH indicator emission signal at a second wavelength and an internal reference emission signal at a third wavelength at a first time point; (c) measuring the pH indicator emission signal at the second wavelength; (d) measuring the internal reference emission signal at the third wavelength; (e) ratiometrically determining the pH in the sample; (f) repeating steps (b)-(e) at least at a second time point; and (g) determining the extracellular acidification rate from the difference in pH in the sample at the first time oint and the at least second time point as a function of time.

In order that this invention be more fully understood, the following examples are set forth. These examples are for the purpose of illustration only and are not to be construed as limiting the scope of the invention in any way.

### EXAMPLES

### Syntheses of Probes

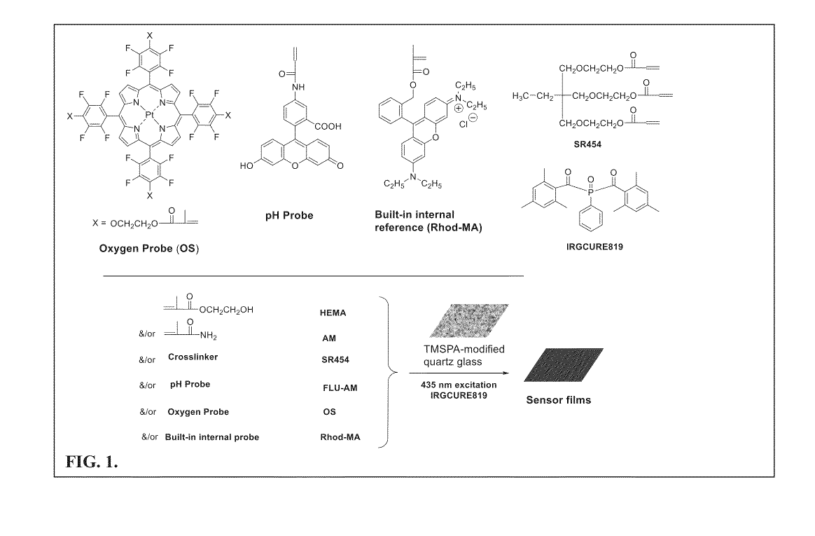

The structures of pfiS and OS are shown in FIG. 1, These probes were synthesized according to previous procedures. [Tian, Y. Q., Fuller, E., Klug, S., Lee, F., Su, F., Zhang, L., Chao, S. H., Metchum, D. R. 2013, Sensors and Actuators B, 188, 1-10; Tian, Y. Q., Shumway, B., Meldrum D. R. 2010, Chemistry of Materials, 22, 2069-2078.].

### pH Sensor Preparation

800 mg HEMA, 150 mg AM, 500 mg SR454, 1 mg Sensor, and 10 mg IRGCURE819 were dissolved in 0.5 mL DMF to make a stock solution for use. 10 μL of the stock solutions were added onto the surface of the 3-acryloxypropyl trimethoxysilane (TMSPA) modified quartz glass and covered with tridecafluorol,1,2,2tetrahydrooctyp trichlorosilane treated cover slip (F treated cover slip) to make a sandwich structure. Using TMSPA to modify the quartz glass was to enable the sensors and matrices to be chemically grafted onto a quartz substrate. The thickness was controlled using 25 μm Kapton tape (DuPont, Wilmingnton, Del.). The sandwich setup was placed under a UV light at 435 nm. After irradiation under the UV light for 50 seconds, the F treated cover slip was removed from the polymerized membrane surface. The polymer membranes on the quartz glasses were washed three times using methanol to remove any remaining nonpolymerized monomers and possible residual solvents or monomers.

### Oxygen Sensor Preparation

800 mg HEMA, 150 mg AM, 500 mg SR454, ling Sensor, and 10 mg IRGCURE819 were dissolved in 0.5 mL DMF to make a stock solution for use. 10 μL of the stock solutions were added onto the surface of the TMSPA modified quartz glass and covered with F-treated cover slip to make a sandwich structure. Using TMSPA to modify the quartz glass was to enable the sensors and matrices to be chemically grafted onto a quartz substrate. The thickness was controlled using 25 μm Kapton tape (DuPont, Wilmington, Del.). The sandwich setup was placed under a UV light at 435 nm. After irradiation under the UV light for 50 seconds, the F-treated cover glass was removed from the polymerized membrane surface. The polymer membranes on the quartz glasses were washed three times using methanol to remove any remaining nonpolymerized monomers and possible residual solvents or monomers.

### Dual pH and Oxygen Sensor Film Preparation

800 mg HEMA, 150 mg AM, 500 mg SR454, 1 mg pH Sensor, 1 mg Rhodamine reference probe, 20 mg of oxygen sensor, and 10 mg IRGCURE819 were dissolved in 0.5 mL DMF to make a stock solution for use. 10 μL of the stock solutions were added onto the surface of the TMSPA modified quartz glass and covered with F-treated cover slip to make a sandwich structure. Using TMSPA to modify the quartz glass was to enable the sensors and matrices to be chemically grafted onto a quartz substrate. The thickness was controlled using 25 μm Kapton tape (DuPont, Wilmington, Del.). The sandwich setup was placed under a UV light at 435 nm. After irradiation under the UV light for 50 seconds, the F-treated cover glass was removed from the polymerized membrane surface. The polymer membranes on the quartz glasses were washed three times using methanol to remove any remaining nonpolymerized monomers and possible residual solvents or monomers.

### pH Responses of the pH Sensor Films

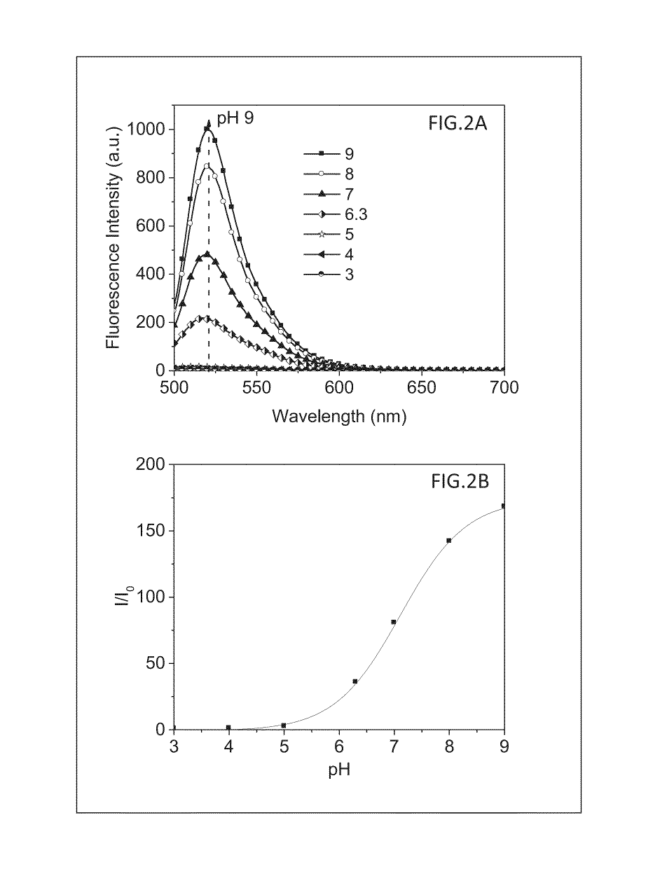

FIG. 2A shows the pH responses of a pH sensor film excited at 488 nm. Its emission intensity with a maximum at 515 nm increases with the increase of pH value. FIG. 2B shows the ratios of the fluorescence intensities at 515 nm at different pH values. It can be found that the fluorescence intensity ratios changed about 175 folds from pH 3 to pH 9, indicating its exceptionally high sensitivity to pH. The sensor has a pKa of 7.1, showing that the sensor is suitable for biological applications. The intramolecular charge transfer and tautomerization of the fluorescein group in the pH sensor results in the pH responses [Tian, Y. Q., Fuller, E., Klug, S., Lee, F., Su, F., Zhang, L., Chao, S. H., Meldrum, D. R. 2013, Sensors and Actuators B, 188, 1-10].

### Oxygen Responses of the Oxygen Sensor Film

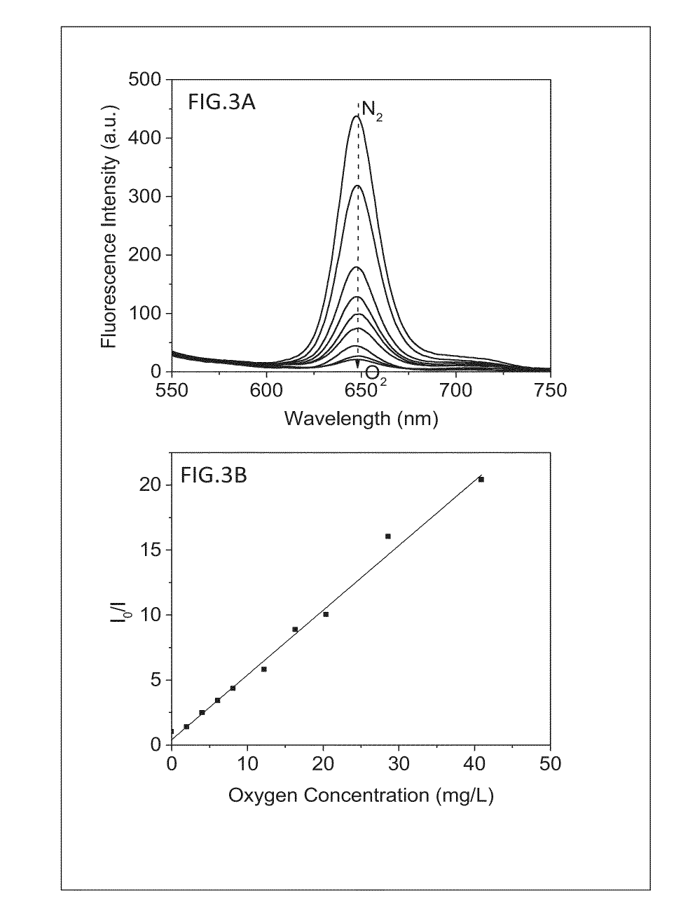

FIG. 3A shows the oxygen responses of the oxygen sensor film. FIG. 3B shows the Stern-Volmer plot of the sensor at different dissolved oxygen concentration. Similar with other oxygen sensor films using the same oxygen probe, linear Stern-Volmer plot was observed.

### pH and Oxygen Responses of the Dual pH and Oxygen Sensor

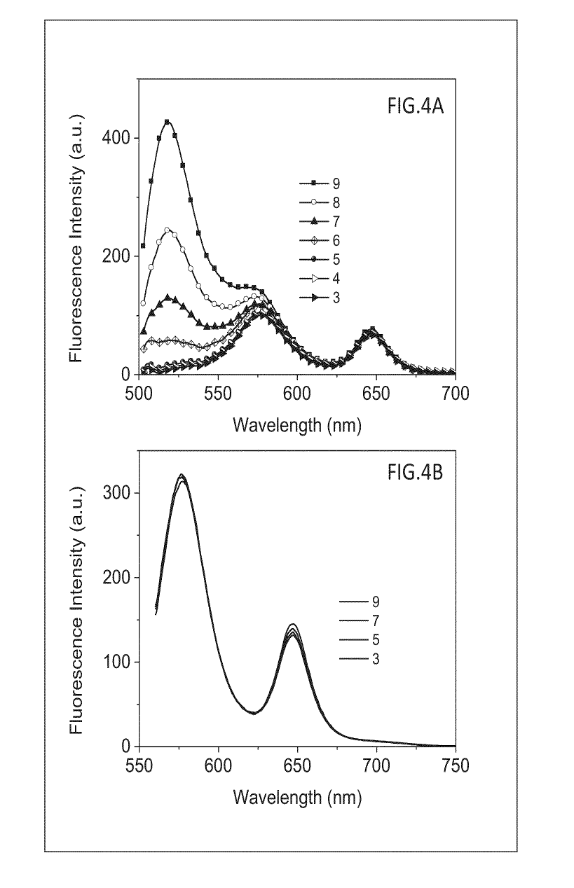

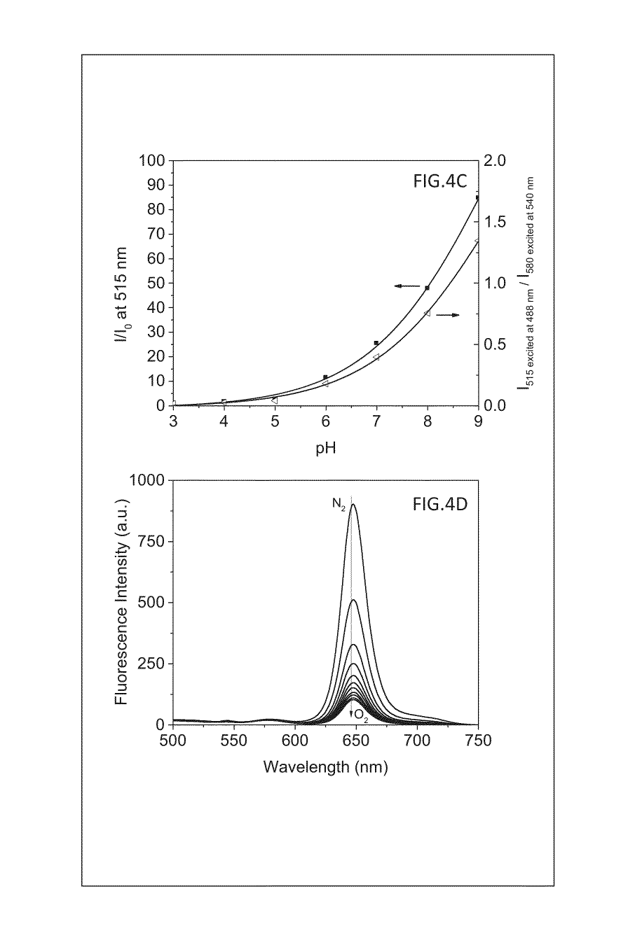

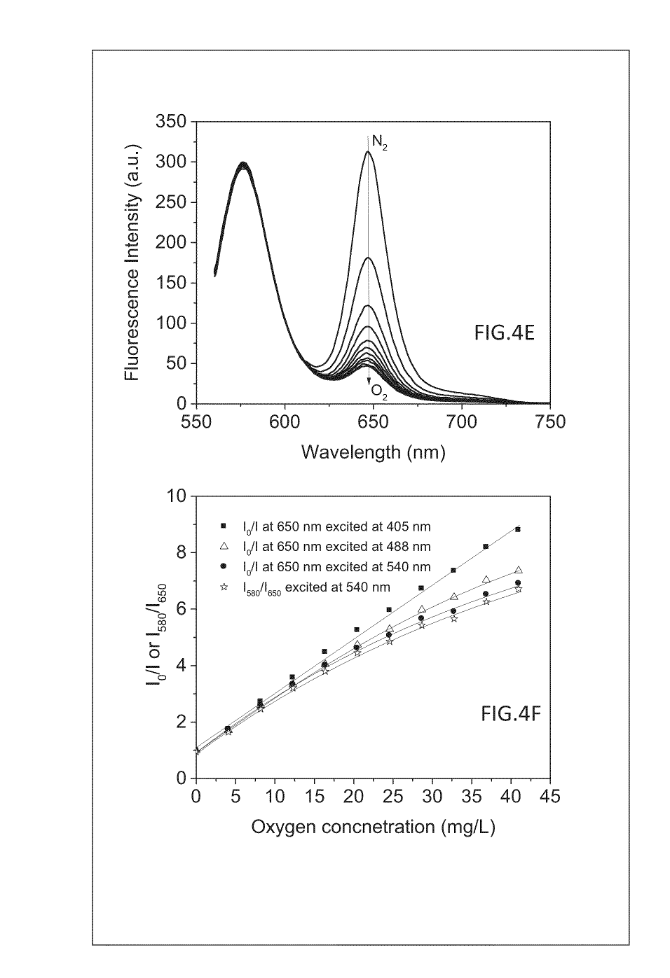

FIG. 4A-FIG. 4F show the pH and oxygen responses of the dual pH and oxygen sensor. The sensor composes a pH probe with an emission maximum at 515 nm, an internal huilt-in reference probe with an emission maximum at 580 nm, and an oxygen probe with an emission maximum at 650 nm. FIG. 4A shows the pH responses of the dual sensor excited at 488 nm. The emission at 515 nm increases with the increase of pH. The emission at 580 nm also increases with the increase of pH when excited at 488 nm. This is due to slightly overlay of the fluorescence from the pH probes with the built-in reference probes. When excited at 540 nm, the emission at 580 nm has no responses to pH (FIG. 4B). The oxygen sensor with an emission maximum at 650 nm does not respond to when excited at either 488 nm or 540 nm. FIG. 4C shows the pH responses of the sensor calculated by the changes of the intensities at 515 nm and also the ratiometric approach using the ratios of emission intensities at 515 nm and at 580 nm. The pH responses cover the physiological ranges from 7.5 to 5.5, indicating its applicability for biological pH measurements. FIG. 4D and FIG. 4E show the oxygen responses excited at 405 nm and 540 nm, respectively. The emission intensities of the oxygen sensor increase with the decreases of dissolved oxygen concentrations, similar to other oxygen sensors. FIG. 4F shows the Stern-Volmer plots of the oxygen responses calculated using different approaches. The sensor responds linearly to oxygen when excited at 405 nm, because at such an excitation wavelength, the rhodamine derived built-in reference and pH probe were not excited efficiently. Although nonlinear Stern-Volmer plots were observed when excited at other wavelengths, such as 488 and 514 nm at high oxygen concentrations, because of the slight overlay of the emissions of the built-in reference probes with the oxygen sensor's emissions, all the plots shows linear responses to oxygen from deoxygenated condition to dissolved oxygen concentration of 10 mg/mL, corresponding to oxygen fraction of 24% in air. The linear responses make the calculation of oxygen concentrations simple when used for cellular oxygen respiration studies.

### Fabrication of 3×3 Dual pH and Oxygen Sensor Arrays

The dual pH and oxygen sensor arrays were photopatterned on fused silica surface either using a standard UV aligner, such as SÜSS MicroTec MA6 or OAI 200, though a chrome mask or using a maskiess photolithography system, such as SF100 system by Intelligent Micro Patterning, LLC), with a AutoCAD virtual mask. The fused silica substrates were activated by 5 min oxygen plasma treatment (Harrick PDC32G, Harrick Plasma, NY) followed by overnight vapor salinization using 3-acryloxypropyl trimethoxysilane for covalent conjugation of sensors. The chrome mask was treated triethoxy-1H,1H,2H,2H-tridecalluoro-n-octylsilane to minimize absorption of sensor material. One example is photopatterned 3×3 arrays of tricolor dual sensors with 50 μm diameter and 300 μm pitch.

### Preparation of the Single Cell Devices for Single Cell Loading, Culture, and Analysis

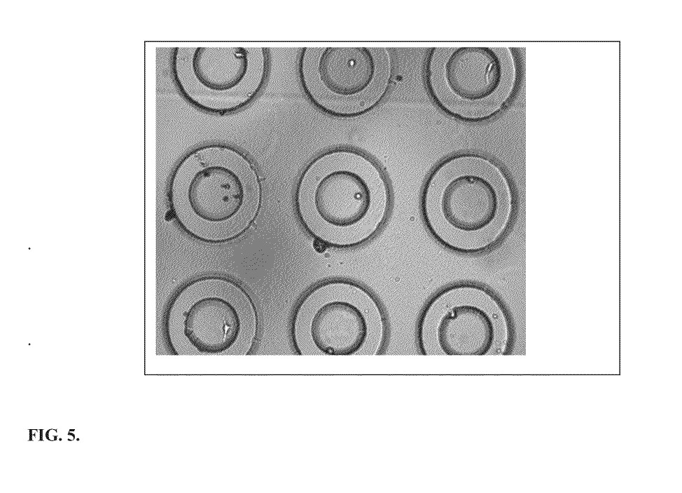

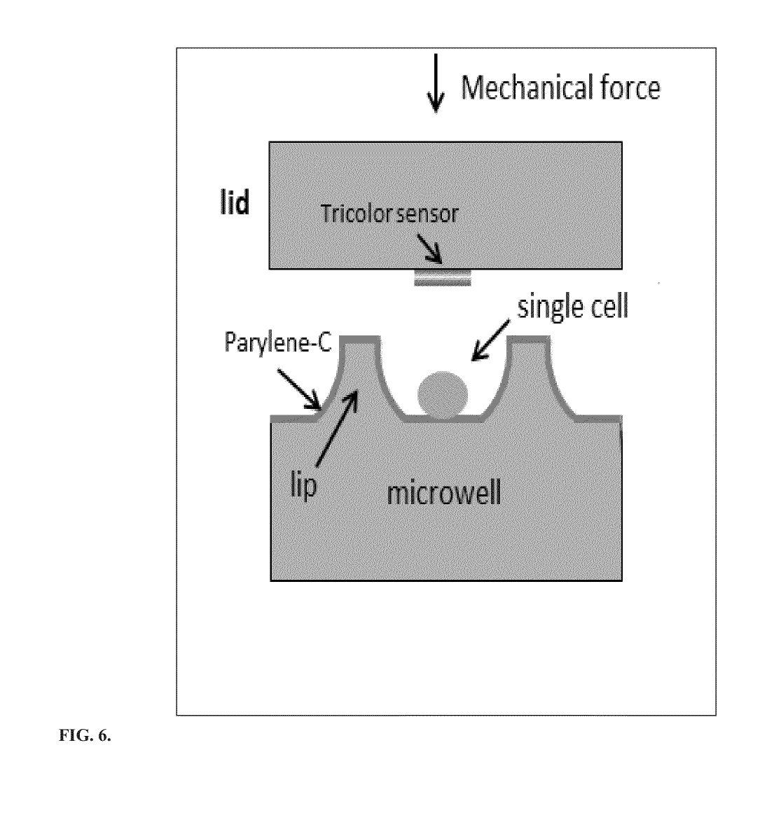

Following our published procedure [Kelbauskas, Ashili, S., Houkal, J., Smith, D., Mohammadreza, A., Lee, K., Kumar, A., Anis, Y., Paulson, T. G., Youngbull, A. C., Tian, Y. Q., Holl, M., Johnson, R. H., Meldrum, D. R. 2012, Journal of Biomedical Optics, 17, 037008 (12 pages)], we loaded microwell arrays with single cells and incubated for 24 hours. One microwell with no cells was used as control as shown in FIG. 5. 24 hours later, the metabolic “drawdown” method was performed by aligning sensor arrays to the microwell arrays on a custom “drawdown” station build around an inverted epi fluorescence microscope (FIG. 6). The fluorescence intensities from the sensor arrays were automatically collected for 120 minutes at 1 minute intervals for collecting the fluorescent signals from the channels for pH probes, oxygen probes, and the Rhodamine reference probes.

### Responses of the Dual pH and Oxygen Sensors in the Sensor Arrays

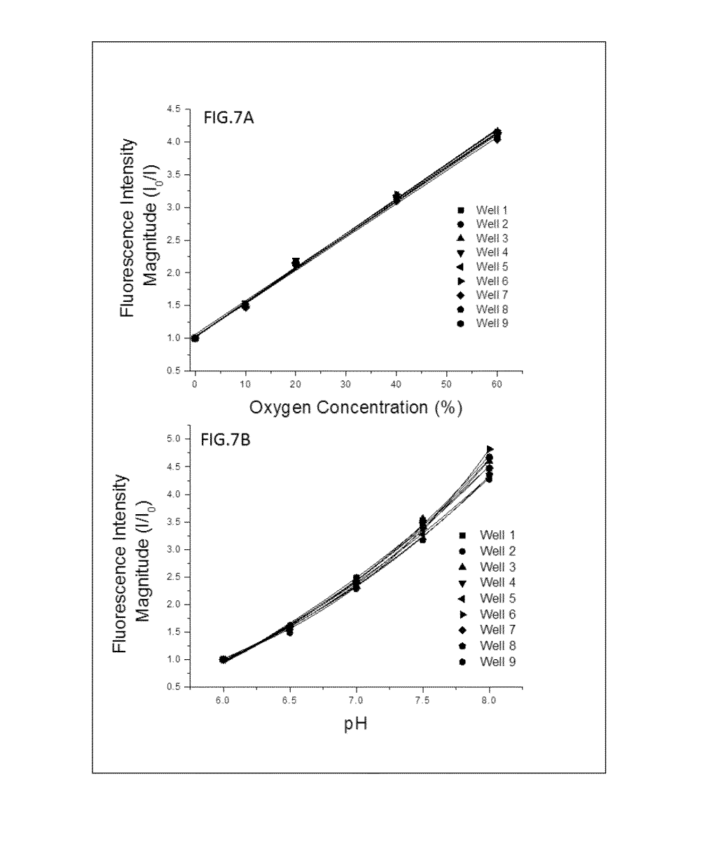

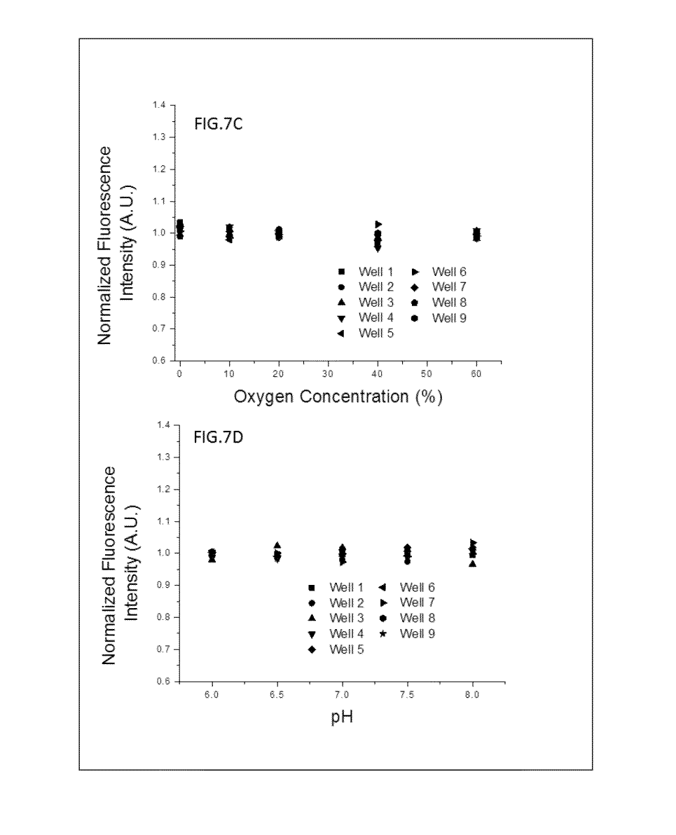

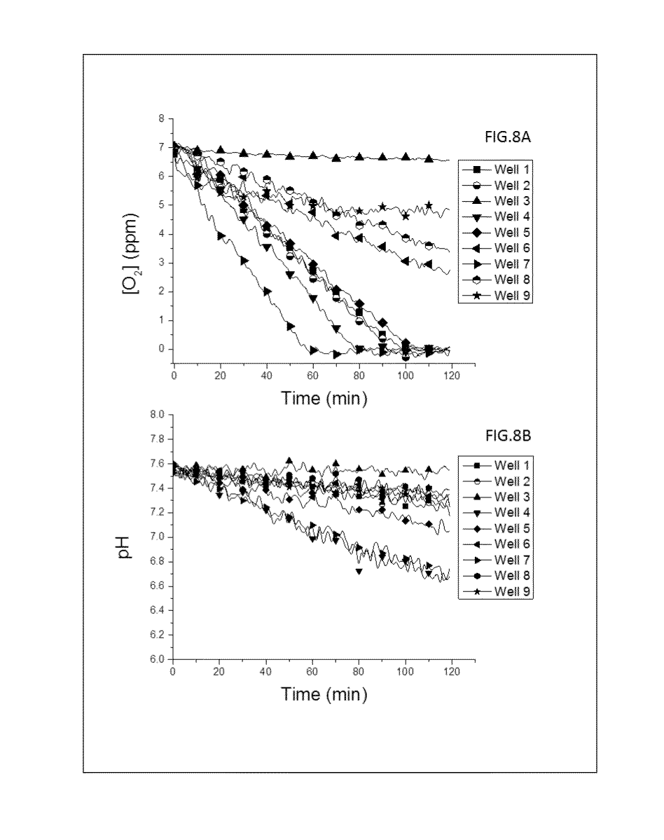

The responses of the sensors in the arrays were measured at different oxygen concentrations and pH values. The results were used as the calibration curves for single cell analysis. FIG. 7A shows the linear Stern-Volmer plot of the oxygen sensors in 9 wells. FIG. 7B shows the pH responses from pH 6 to 8. It was also found that the rhodamine reference probes are not affected by oxygen concentrations or pH values (FIG. 7C and 7D). FIG. 8A and FIG. 8B showed the time dependent changes of oxygen concentrations and pH values of single cells (immortalized human metaplastic esophageal epithelial cells) in 8 wells with one empty well used as a reference. Clearly, heterogeneous oxygen consumption rates and extracellular acidification rates were observed.

While particular materials, formulations, operational sequences, process parameters, and end products have been set forth to describe and exemplify this invention, they are not intended to be limiting. Rather, it should be noted by those ordinarily skilled in the art that the written disclosures are exemplary only and that various other alternatives, adaptations, and modifications may be made within the scope of the present disclosure.

What is claimed is:

1. An optical luminescence dual sensor comprising a copolymer, wherein the copolymer comprises:
(a) a polymerized form of a probe for sensing pH;
(b) a polymerized form of a probe for sensing oxygen;
(c) a polymerized form of an internal reference probe; and
(d) a matrix comprising a polymer selected from the group consisting of poly(2-hydroxyethyl methacrylate) (PHEMA), polyacrylamide (PAM), poly(poly(2-(2-(2-methoxyethoxy)ethoxy)ethyl methacrylate)) (POEGMA), poly(N-isopropyl acrylamide) (PNIPAAm), and copolymers thereof;
wherein:
the probe for sensing pH has formula (I): 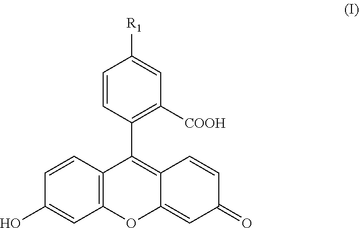
wherein
R₁ is CₘH₂ₘX or NHCOCₘH₂ₘY, where m is an integer selected from the group consisting of 0, 1, 2, 3, 4, 5, 6, 8 and 11;
X is selected from the group consisting of: 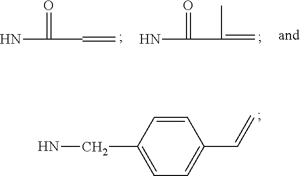
and
Y is selected from the group consisting of: 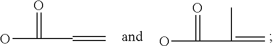
(b) the probe for sensing oxygen has formula (II): 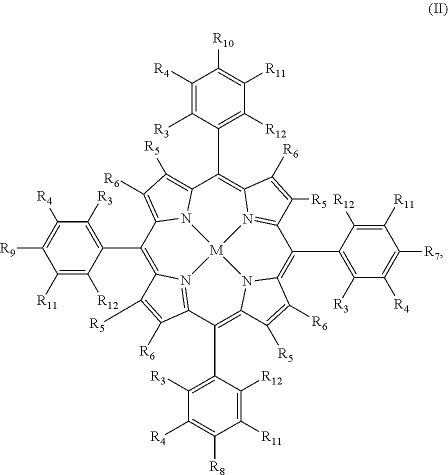
where M is selected from Pt or Pd;
R₁₁ and R₁₂ can be the same or different and are independently selected from the group consisting of H, halo, CH₃, OCH₃ and OC₂H₅;
R₃ and R₄ can be the same or different and are independently selected from the group consisting of H, halo, CH₃, OCH₃ and OC₂H₅;
R₅ and R₆ can be the same or different and are independently selected from the group consisting of H, halo, CH₃, OCH₃ and OC₂H₅;
R₇, R₈, R₉ and R₁₀ can be the same or different and are independently selected from the group consisting of (CH₂)ₚOH, O(CH₂)ₚOH, NH(CH₂)ₚOH, (CH₂)ₚOM′A, O(CH₂)ₚOM′A, NH(CH₂)ₚOM′A, (CH₂)ₚOA, O(CH₂)ₚOA, NH(CH₂)ₚOA, (CH₂)ₚOVA, O(CH₂)ₚOVA, NH(CH₂)OVA, (OCH₂CH₂)_{q}OH, NH(CH₂CH₂O)_{q}H, (OCH₂CH₂)_{q}OM′A, NH(CH₂CH₂O)_{q}M′A, (OCH₂CH₂)_{q}OH, NH(CH₂CH₂O)_{q}A, (OCH₂CH₂)_{q}OVA, NH(CH₂CH₂O)_{q}VA,
where
M′A is 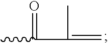
A is 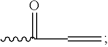
VA is 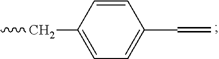
p is an integer selected from the group of consisting of 0, 1, 2, 3, 4, 5, 6, 7, 8, 9, 10, 11 and 12; and
c is an integer selected from the group of consisting of 1, 2, 3, 4, 5, 6, 7, 8, 9, 10, 11, 12, 13, 14, 15, 16, 17, 18, 19, 20, 21, 22, 23, 38, 39, 40, 41, 42, 43, 44, 140, 141, 142, 143, 144, 145, 146, 147, 148, 149 and 150; and
(c) the internal reference probe has formula (III): 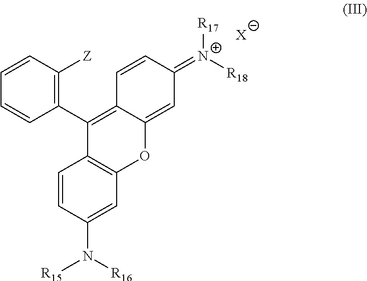
wherein R₁₅, R₁₆, R₁₇, and R₁₈ can be the same or different and are independently CₙH₂ₙ₊₁, where n is an integer selected from the group consisting of 1, 2, 3, 4, 5, 6, 7 and 8;
X is an anion;
Z is selected from the group consisting of: (CH₂)ₚOH, O(CH₂)ₚOH, NH(CH₂)ₚOH, (CH₂)ₚOM′A, O(CH₂)ₚOM′A, NH(CH₂)ₚOM′A, (CH₂)ₚOA, O(CH₂)ₚOA, NH(CH₂)ₚOA, (CH₂)ₚOVA, O(CH₂)ₚOVA, NH(CH₂)ₚOVA, (OCH₂CH₂)_{q}OH, NH(CH₂CH₂O)_{q}H, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA, NH(CH₂CH₂O)_{q}VA, CH₂(OCH₂CH₂)ᵣOA, CH₂(OCH₂CH₂)ᵣOM′A, CH₂(OCH₂CH₂)ᵣOVA; and
r is an integer selected from the group of consisting of 1, 2, 3, 4, 5, 6, 7, 8, 9, 10, 11, 12, 13, 14, 15, 16, 17, 18, 19, 20, 21, 22, 23, 38, 39, 40, 41, 42, 43, 44, 140, 141, 142, 143, 144, 145, 146, 147, 148, 149 and 150.

2. The optical luminescence dual sensor of claim 1, wherein, in the probe for sensing pH, R₁ is CₘH₂ₘX, where m is 0.

3. The optical luminescence dual sensor of claim 1, wherein, in the probe for sensing pH, X is

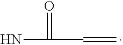

4. The optical luminescence dual sensor of claim 1, wherein the probe for sensm pH is:

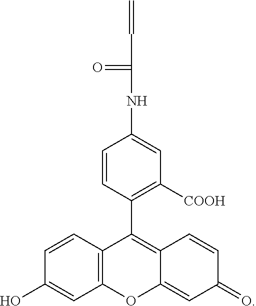 [SMILES: C=CC(=O)Nc1ccc(-c2c3ccc(=O)cc-3oc3cc(O)ccc23)c(C(=O)O)c1]

5. The optical luminescence dual sensor of claim 1, wherein, in the probe for sensing oxygen, R₃, R₄, R₅, R₆, R₁₁ and R₁₂ are F or H.

6. The optical luminescence dual sensor of claim 1, wherein, in the probe for sensing oxygen, M is Pt.

7. The optical luminescence dual sensor of claim 1, wherein, in the probe foreensing oxygen, R₇, R₈, R₉ and R₁₀ are O(CH₂)ₚOM′A and p is 2.

8. The optical tut inescence dual sensor of claim 1, wherein the probe for sensing oxygen is: 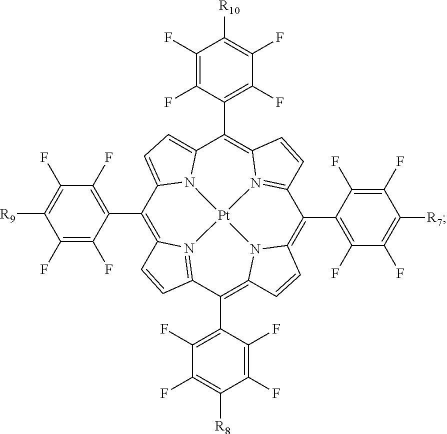
and
R₇, R₈, R₉ and R₁₀ are

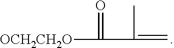

9. The optical luminescence dual sensor of claim 1, wherein, in the internal reference probe, R₁₅, R₁₆, R₁₇, R₁₈ are CₙH₂ₙ₊₁, where n is 2.

10. The optical tut inescence dual sensor of claim 1, wherein, in the internal reference probe, X is halo.

11. The optical luminescence dual sensor of claim 1, wherein, in the internal reference probe, Z is (CH₂)ₚOM′A and p is 1.

12. The optical luminescence dual sensor of claim 1, wherein the internal reference probe is:

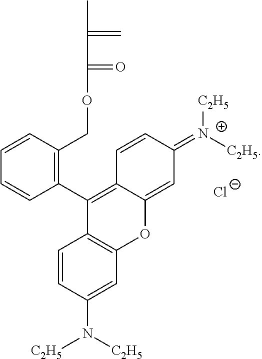 [SMILES: C=C(C)C(=O)OCc1ccccc1-c1c2ccc(=[N+](CC)CC)cc-2oc2cc(N(CC)CC)ccc12 ; [Cl-]]

13. A method of preparing an optical luminescence dual sensor, wherein the method comprises the steps of:
(a) copolymerizing a probe for sensing pH, a probe for sensing oxygen, and an internal reference probe, with polyacrylamide and poly(2-hydroxyethyl methacrylate)-co-polyacrylamide in the presence of a crosslinker and an initiator;
wherein the probe for sensing pH has formula (I): 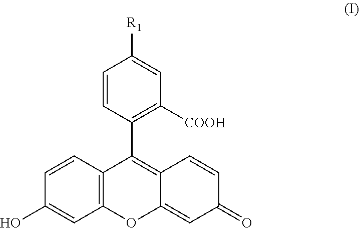
wherein
R₁ is CₘH₂ₘX or NHCOCₘH₂ₘY, where m is an integer selected from the group consisting of 0, 1, 2, 3, 4, 5, 6, 8 and 11;
X is selected from the group consisting of: 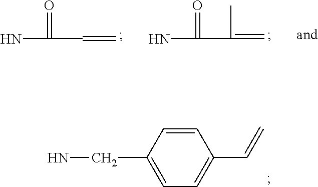
and
Y is selected from the group consisting of: 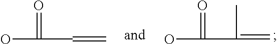
the probe for sensing oxygen has formula (II): 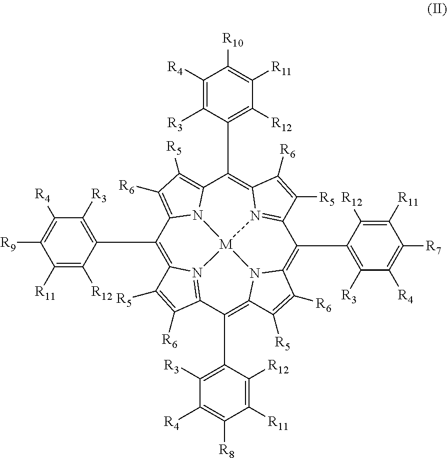
where M is selected from Pt or Pd;
R₁₁ and R₁₂ can be the same or different and are independently selected from the group consisting of H, halo, CH₃, OCH₃ and OC₂H₅;
R₃ and R₄ can be the same or different and are independently selected from the group consisting of H, halo, CH₃, OCH₃ and OC₂H₅;
R₅ and R₆ can be the same or different and are independently selected from the group consisting of H, halo, CH₃, OCH₃ and OC₂H₅;
R₇, R₈, R₉ and Rio can be the same or different and are independently selected from the group consisting of (CH₂)ₚOH, O(CH₂)ₚOH, NH(CH₂)ₚOH, (CH₂)ₚOM′A, O(CH₂)ₚOM′A, NH(CH₂)ₚOM′A, (CH₂)ₚOA, O(CH₂)ₚOA, NH(CH₂)ₚOA, (CH₂)ₚOVA, O(CH₂)ₚOVA, NH(CH₂)ₚOVA, (OCH₂CH₂)_{q}OH, NH(CH₂CH₂O)_{q}H, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA, NH(CH₂CH₂O)_{q}VA,
where
M′A is 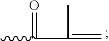
A is 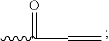
VA is 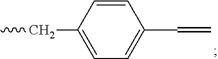
and
p is an integer selected from the group of consisting of 0, 1, 2, 3, 4, 5, 6, 7, 8, 9, 10, 11 and 12.
q is an integer selected from the group of consisting of 1, 2, 3, 4, 5, 6, 7, 8, 9, 10, 11, 12, 13, 14, 15, 16, 17, 18, 19, 20, 21, 22, 23, 38, 39, 40, 41, 42, 43, 44, 140, 141, 142, 143, 144, 145, 146, 147, 148, 149 and 150; and
the internal reference probe has formula (III): 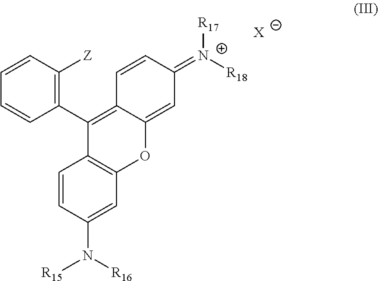
wherein R₁₅, R₁₆, R₁₇, and R₁₈ can be the same or different and independently are CₙH₂ₙ₊₁, where it is an integer selected from the group consisting of 1, 2, 3, 4, 5, 6, 7 and 8;
X is an anion; and
Z is selected from the group consisting of: (CH₂)ₚOH, O(CH₂)ₚOH, NH(CH₂)ₚOH, (CH₂)ₚOM′A, O(CH₂)ₚOM′A, NH(CH₂)ₚOM′A, (CH₂)ₚOA, O(CH₂)ₚOA, NH(CH₂)ₚOA, (CH₂)ₚOVA, O(CH₂)ₚOVA, NH(CH₂)ₚOVA, (OCH₂CH₂)_{q}OH, NH(CH₂CH₂O)_{q}H, (OCH₂CH₂)_{q}OM′A, NH(CH₂CH₂O)_{q}M′A, (OCH₂CH₂)_{q}OA, NH(CH₂CH₂O)_{q}A, (OCH₂CH₂)_{q}OVA, NH(CH₂CH₂O)_{q}VA, CH₂(OCH₂CH₂)ᵣOA, CH₂(OCH₂CH₂)ᵣOM′A, CH₂(OCH₂CH₂)ᵣOVA; and
r is an integer selected from the group of consisting of 1, 2, 3, 4, 5, 6, 7, 8, 9, 10, 11, 12, 13, 14, 15, 16, 17, 18, 19, 20, 21, 22, 23, 38, 39, 40, 41, 42, 43, 44, 140, 141, 142, 143, 144, 145, 146, 147, 148, 149 and 150
and
(b) immobilizing or attaching the copolymer of step substrate.

14. The method of claim 13,
wherein the probe for sensing pH is: 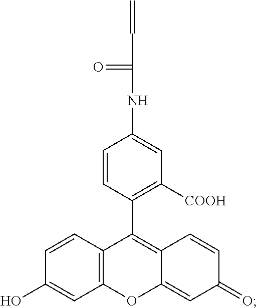 [SMILES: C=CC(=O)Nc1ccc(-c2c3ccc(=O)cc-3oc3cc(O)ccc23)c(C(=O)O)c1]
the probe for sensing oxygen is: 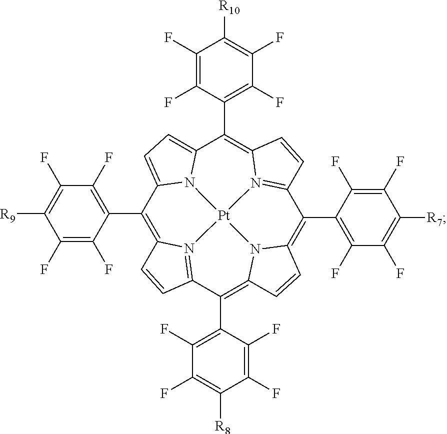
R₇, R₈, R₉ and R₁₀ are 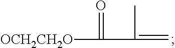
and
the internal reference probe is:

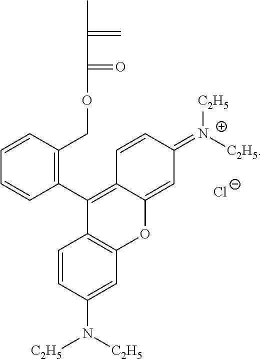 [SMILES: C=C(C)C(=O)OCc1ccccc1-c1c2ccc(=[N+](CC)CC)cc-2oc2cc(N(CC)CC)ccc12 ; [Cl-]]

15. A method of preparing a dual pH and oxygen array on a substrate, wherein the method comprises:
(a) masking the substrate to define boundaries on the substrate;
(b) contacting the unmasked substrate with a conjugate compound to form a conjugated layer-substrate;
(c) contacting the conjugated layer-substrate with the sensor of claim 1.

16. A method of deter ining pH of a sample, wherein the method comprises:
(a) exposing the sample to an optical luminescence dual sensor according to claim 1;
(b) irradiating the sensor at a first wavelength to produce a indicator emission signal at a second wavelength and an internal reference emission signal at a third wavelength;
(c) measuring the pH indicator emission signal at the second wavelength;
(d) measuring the internal reference emission signal at the third emission wavelength; and
(e) ratiometrically determining the pH of the sample.

17. A method of determining oxygen concentration in a sample, wherein the method comprises:
(a) exposing the sample to an optical luminescence dual sensor according to claim 1;
(b) irradiating the sensor at a first wavelength to produce an oxygen indicator emission signal at a second wavelength and an internal reference emission signal at a third wavelength;
(c) measuring the oxygen indicator emission signal at the second wavelength;
(d) measuring the internal reference emission signal at the third wavelength; and
(e) ratiometrically determining the oxygen concentration in the sample.

18. A method of simultaneously determining pH and oxygen concentration in a sample, Wherein the method comprises:
(a) exposing the sample to an optical luminescence dual sensor according to claim 1;
(b) irradiating the sensor (i) at a first wavelength to produce a pH indicator emission signal at a second wavelength, (ii) at a third wavelength to produce an oxygen indicator emission signal at a fourth wavelength and (iii) at a fifth wavelength to produce an internal reference emission signal at a sixth wavelength;
(c) measuring the pH indicator emission signal at the second wavelength;
(d) measuring the oxygen indicator emission signal at the fourth wavelength;
(e) measuring the internal reference emission signal at the sixth wavelength;
(f) ratiometrically determining the pH of the sample using the measurements obtained in steps (c) and (e); and
(g) ratiometrically determining the oxygen concentration of the sample using the measurements obtained in steps (d) and (e).

19. A method of detecting single cell respiration, wherein the method comprises:
(a) exposing the cell to an optical luminescence dual sensor according to claim 1;
(b) irradiating the sensor at a first wavelength to produce an oxygen indicator emission signal at a second wavelength and an internal reference emission signal at a third wavelength at a first time point;
(c) measuring the oxygen indicator emission signal at the second wavelength;
(d) measuring the internal reference emission signal at the third wavelength;
(e) ratiometrically determining the oxygen concentration in the sample; and
(f) repeating steps (b)-(e) at least at a second time point,
wherein an increase in the oxygen concentration at the at least second time point indicates cell respiration.

20. A method of detecting extracellularacidification in a sample, wherein the method comprises:
(a) exposing the sample to an optical luminescence dual sensor according to claim 1;
(b) irradiating the sensor at a first wavelength to produce a pH indicator emission signal at a second wavelength and an internal reference emission signal at a third wavelength at a first time point;
(c) measuring the pH indicator emission signal at the second wavelength;
(d) measuring the internal reference emission signal at the third wavelength;
(e) ratiometrically determining the pH in the sample; and
(f) repeating steps (b)-(e) at least at a second time point,
wherein a decrease in the pH at the at least second time point indicates extracellular acidification.
